(19) United States
(12) Patent Application Publication
(10) Pub. No.: US 2004/0093169 A1
(43) Pub. Date: May 13, 2004

(54) Method of designing molecular structure of enzyme inhibitor

(75) Inventors: Yoshiyuki Mizushina, Noda-shi (JP); Kengo Sakaguchi, Tsukuba-shi (JP); Fumio Sugawara, Niiza-shi (JP)

Correspondence Address: FRISHAUF, HOLTZ, GOODMAN & CHICK, PC, 767 THIRD AVENUE, 25TH FLOOR, NEW YORK, NY, 10017-2023, US

(73) Assignee: TOYO SUISAN KAISHA, LTD., Tokyo (JP)

(21) Appl. No.: 10/448,871

(22) Filed: May 29, 2003

Related U.S. Application Data
(63) Continuation of application No. PCT/JP00/08467, filed on Nov. 30, 2000.

(51) Int. Cl. G06F 19/00; G01N 33/48; G06G 7/48; A01N 37/18; A01N 43/04

(52) U.S. Cl. 702/27; 703/11; 702/19; 514/2; 514/44

(57) ABSTRACT

A method of designing a molecular structure of an inhibitor of a target enzyme by analyzing, in the tertiary structure of a first enzyme, a geometric configuration formed by amino acids to which an inhibitor on the first enzyme is to bind. The method comprises (1) a step of determining the amino acid group of the first enzyme to which the inhibitor is to bind, (2) a step of determining the geometric configuration of the amino acid group of the step (1), (3) a step of searching for an amino acid group having a geometric configuration similar to that of the step (2), in the target enzyme, and (4) a step of searching weather or not (i) the amino acids constituting the amino acid group are present at the surface of the target enzyme, and (ii) the amino acids have been conserved in different species by comparison with an enzyme derived from an organism of different types from the organism from which the target enzyme is derived. When the requirements are satisfied, it is assumed that the geometric configuration formed by amino acids to which the inhibitor of the first enzyme is to bind, is also present in the target enzyme, then the molecular structure of an inhibitor of the target enzyme is designed.

### CROSS-REFERENCE TO RELATED APPLICATIONS

This is a Continuation Application of PCT Application No. PCT/JP00/08467, filed Nov. 30, 2000, which was not published under PCT Article 21(2) in English.

### BACKGROUND OF THE INVENTION

1. Field of the Invention

The present invention relates to a method of designing an enzyme inhibitor.

More specifically, the present invention relates to a method of designing a molecular structure of an inhibitor on a target enzyme, the secondary and tertiary structures of which being known, comprising: analyzing, in the tertiary structure of a first enzyme whose primary, secondary and tertiary structures are known, a geometric configuration formed by amino acids to which a first inhibitor, having biochemical, inhibitory activity on the first enzyme, is to bind; and searching, in the target enzyme, the similar geometric configuration to the geometric configuration formed in the first enzyme, thereby designing a molecular structure of an inhibitor on the target enzyme.

Further, the present invention relates to another method of designing a molecular structure of an inhibitor on a target enzyme whose tertiary structure is unknown, comprising: analyzing, in the tertiary structure of a first enzyme whose primary, secondary and tertiary structures are known, a geometric configuration formed by amino acids to which a first inhibitor having biochemical, inhibitory activity on the first enzyme, is to bind; and searching, in a second enzyme whose primary, secondary and tertiary structures are known, the similar geometric configuration to the geometric configuration formed in the first enzyme, thereby designing the molecular structure of the inhibitor on the target enzyme.

2. Description of the Related Art

DNA is a substance which carry genetic information. It is well known that “DNA replication” as a reaction in which DNA is doubled, “DNA repair” as a reaction in which errors and damages in the structure thereof are repaired, and “DNA recombination” as a reaction in which genetic information is exchanged, are the basis of all phenomena of living organism. A series of DNA synthesis reactions as described above are carried out mainly by the enzymes called DNA polymerase.

As DNA polymerase (hereinafter also referred to as “pol.”) of eucaryote (mainly mammals), there have been discovered 8 types of DNA polymerase, which are identified as pol. α, β, γ, δ, ε, ζ, η and θ, according to the molecular weight, features in terms of protein-chemistry and differences in a function of DNA synthesis (1-4) (Note that the number in the parenthesis represents the number of the references listed in the reference section described below). It is assumed that these enzymes play a variety of roles, respectively. Specifically, pol. α, δ and ε are presumably involved in DNA replication of nucleus (1), pol. γ is presumably involved in DNA replication of mitochondria (1), and pol. β, ζ, η and θ are presumably involved in repair of nucleus DNA (1-4) Further, it has been suggested that pol. β is also involved in DNA recombination (1). However, biochemical roles of DNA polymerase in DNA synthesis reactions, are still unclear in many aspects.

Well-known examples of a molecular biological technique, which is often employed in the study of the roles played by enzymes, generally include: a method of producing a mutant having deficiency of the enzyme, and thereby investigating the phenotype; or a method of producing an individual whose gene encoding the enzyme has been knocked-out, thereby investigating the phenotype. However, in the case of DNA polymerase, application of such methods as described above most likely results in the death of the organism. Thus, it is difficult to analyze the roles of the enzyme by using such methods.

On the other hand, there has been another approach in which the functions of the DNA polymerase are studied on the basis of the analysis of an inhibitor on the enzyme. Conventionally, an inhibitor on an enzyme is searched by using natural substances, chemical files and the like. However, such conventional methods are basically the methods in which an inhibitor is discovered simply “by chance” after an enormous number of tests are carried out, which is by no means efficient.

If the structure of the amino acid residues constituting an enzyme can be identified up to the tertiary level thereof, the molecular structure of an inhibitor on the enzyme will more easily be deduced. However, an enzyme generally has a large molecular weight and a relatively complicated three-dimensional structure, and it is thus quite often difficult to analyze the tertiary structure thereof.

In consideration of the problems described above, an object of the present invention is to provide a method of efficiently discovering an inhibitor on an enzyme without carrying out a number of trial and error. Particularly, the object of the present invention is to provide a method of efficiently discovering an inhibitor on an enzyme whose molecular weight is approximately 30,000 or more, and thus, the tertiary structure of which cannot be identified with NMR, without carrying out a number of trial and error.

### BRIEF SUMMARY OF THE INVENTION

The inventors of the present invention have discovered as a result of intense study, a method of efficiently designing molecular structure of an inhibitor on an enzyme to complete the present invention. The method comprises identifying the amino acid residues of a specific enzyme, to which residues an inhibitor is to bind in an interaction between the enzyme and the inhibitor, identifying the geometric configuration formed by the Cα atoms of each of the amino acid residues, and analyzing by search (1) whether or not these amino acids are those present at the surface of the enzyme protein and (2) whether or not these amino acids are those which have been conserved in terms of the evolution theory. By the method, the number of tests required for designing a molecular structure of an inhibitor on an enzyme can be significantly reduced, as compared with the conventional method where the inhibitor is searched at random.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate presently preferred embodiments of the invention, and together with the general description given above and the detailed description of the preferred embodiments given below, serve to explain the principles of the invention.

FIG. 1 is a block diagram showing the scheme of each process of a first method of the present invention;

FIG. 2A is a view showing the amino acid sequence of rat pol. β. FIG. 2B is a view showing the amino acid sequence of enzyme DNA topoisomerase II (hereinafter also referred to as “topo II”);

FIG. 3 is a view of a three-dimensional model showing the amino acid chain of rat pol. β;

FIG. 4 is a block diagram showing the scheme of each process of a second method of the present invention;

FIG. 5 is a graph showing the inhibitory activity of nervonic acid on rat pol. β;

FIG. 6 is ¹H-¹⁵N HMQC spectroscopy of rat pol. β;

FIG. 7 is a bar graph showing shift values of ¹H HMQC spectroscopy of rat pol. β;

FIG. 8 is a bar graph showing shift values of ¹⁵N HMQC spectroscopy of rat pol. β;

FIG. 9A is a view showing the geometric configuration of the amino acid residues of rat pol. β, which residues are to be bound to nervonic acid. FIG. 9B is a view showing the geometric configuration of the amino acid residues of yeast topo II, which residues are to be bound to nervonic acid;

FIG. 10A is a view showing a three-dimensional model of rat pol. β, in which the position of the amino acid residues, which residues are to be bound to nervonic acid, is clearly indicated. FIG. 10B is a view showing a three-dimensional model of yeast topo II, in which the position of the amino acid residues, which residues are to be bound to nervonic acid, is clearly indicated;

FIGS. 11A-11F are views showing the primary sequence of amino acids of 31 types of enzymes used in the search of example 1;

FIG. 12A is a view showing the geometric configuration of the amino acid residues of rat pol. β, which residues are to be bound to nervonic acid. FIG. 12B is a view showing the geometric configuration of the amino acid residues of human immunodeficiency virus (HIV) reverse transcriptase, which residues are to be bound to nervonic acid;

FIG. 13A is a view showing a three-dimensional model of rat pol. β, in which the position of the amino acid residues, which residues are to be bound to nervonic acid, is clearly indicated. FIG. 13B is a view showing a three-dimensional model of HIV reverse transcriptase, in which the position of the amino acid residues, which residues are to be bound to nervonic acid, is clearly indicated;

FIG. 14A is a view showing a model in a state in which nervonic acid has bound to the 8 kDa domain of rat pol. β. FIG. 14B is a view in which the model of FIG. 14A has been turned by 90° around the vertical axis;

FIG. 15 is a graph showing the inhibitory activity of nervonic acid on rat DNA polymerase, HIV reverse transcriptase and the like;

FIG. 16 is a view showing the result of electrophoresis, which represents the inhibitory activity effected by nervonic acid on yeast topo II;

FIG. 17 is a view showing the primary sequence of amino acids of yeast topo II or the like, in which the amino acid residues which have been conserved, in terms of the evolution theory, are indicated;

FIG. 18 shows ¹H-¹⁵N HMQC spectroscopy of rat pol. β;

FIG. 19 is a bar graph showing shift values of ¹H HMQC spectroscopy of rat pol. β;

FIG. 20 is a bar graph showing shift values of ¹⁵N HMQC spectroscopy of rat pol. β;

FIG. 21A is a view showing the geometric configuration of the amino acid residues of rat pol. β, which residues are to be bound to lithocholic acid. FIG. 21B is a view showing the geometric configuration of the amino acid residues of yeast topo II, which residues are to be bound to lithocholic acid;

FIG. 22A is a view showing a three-dimensional model of rat pol. β, in which the position of the amino acid residues, which residues are to be bound to lithocholic acid, is clearly indicated. FIG. 22B is a view showing a three-dimensional model of yeast topo II, in which the position of the amino acid residues, which residues are to be bound to lithocholic acid, is clearly indicated; and

FIG. 23 is a view showing the primary sequence of amino acids of yeast topo II or the like, in which the amino acid residues which have been conserved, in terms of the evolution theory, are indicated.

### DETAILED DESCRIPTION OF THE INVENTION

According to one aspect of the present invention, there is provided a method of designing a molecular structure of an inhibitor on an enzyme whose secondary and tertiary structures are known. Hereinafter, this method will be referred to as “the first method of the present invention”.

Specifically, the first method of the present invention is a method of designing a molecular structure of an inhibitor on a target enzyme whose secondary and tertiary structures are known, comprising:

analyzing, in the tertiary structure of a first enzyme whose primary, secondary and tertiary structures are known, a geometric configuration formed by amino acids to which a first inhibitor having biological, inhibitory activity on the first enzyme, is to bind; and

searching, in the target enzyme, amino acids constituting the similar geometric configuration to the geometric configuration that was analyzed in the first enzyme;

thereby designing the molecular structure of the inhibitor on the target enzyme.

In more detail, the first method of the present invention is a method of designing a molecular structure of an inhibitor on a target enzyme whose secondary and tertiary structures are known, comprising:

(1) a step of determining, in the tertiary structure f a first enzyme, a first pocket amino acid group consisting of plural amino acids to which a first inhibitor is to bind, wherein the plural amino acids are determined by comparing the ¹H-¹⁵N HMQC spectroscopy of the first enzyme in the absence of the first inhibitor with the ¹H-¹⁵N HMQC spectroscopy of the first enzyme in the presence of the first inhibitor, and selecting amino acids that satisfy the condition that the absolute value of ¹H chemical shift is equal to or more than a specific value and/or the absolute value of ¹⁵N chemical shift is equal to or more than another specific value;

(2) a step of determining the geometric configuration of the first pocket amino acid group, by measuring, in the tertiary structure of the first enzyme, the distances between the Cα atoms of each of the plural amino acids constituting the first pocket amino acid group;

(3) a step of detecting, in the tertiary structure of the target enzyme, one or more candidates for a target pocket amino acid group having a geometric configuration similar to the geometric configuration formed by the first pocket amino acid group determined in the step (2), wherein it is presumed that the candidate for the target pocket amino acid group is present in the target enzyme if the following conditions are satisfied:

respective amino acids constituting the candidate for the target pocket amino acid group are of the same kinds as the plural amino acids constituting the first pocket amino acid group determined in the step (2), and

the absolute values of differences between (a) respective distances, in the tertiary structure of the amino acids of the target enzyme, between the Cα atoms of each of the amino acids constituting the target pocket amino acid group, and (b) the respective distances between the Cα atoms in the first pocket amino acid group obtained in the step (2) are within a specific value; and

(4) a step of screening the amino acids constituting the candidate for the target pocket amino acid group by the following two requirements:

(requirement 1) the amino acids are those present at the surface of the target enzyme in the tertiary structure thereof; and

(requirement 2) the amino acids are those that have been conserved among species, when a comparison is made between the primary sequence of the amino acids of the target enzyme and the primary sequence of the amino acids of another enzyme having the same biochemical activity as the target enzyme but derived from an organism of a different species from that of the target enzyme, in terms of biological classification; and

determining the amino acids satisfying the above two requirements to be those constituting the usable target pocket amino acid group for designing the molecular structure of the inhibitor on the target enzyme.

In the first method of the present invention, the molecular structure of the inhibitor on the target enzyme is designed on the basis of the kinds of amino acid residues of the first enzyme, to which the first inhibitor is to bind, and the geometric configuration of amino acid residues.

An example of carrying out the first method of the present invention mentioned above is as follows. However, the first method of the present invention is not limited to this.

A method of designing a molecular structure of an inhibitor on human immunodeficiency virus (HIV) reverse transcriptase by analyzing, in the tertiary structure of rat DNA polymerase β, a geometric configuration formed by amino acids to which nervonic acid is to bind; searching, in HIV reverse transcriptase, the similar geometric configuration to the geometric configuration analyzed in rat DNA polymerase β; thereby designing the molecular structure of the inhibitor on HIV reverse transcriptase on the basis of the kinds of amino acid residues of rat DNA polymerase β, to which nervonic acid is to bind, and the geometric configuration of the amino acid residues.

In more detail, the example of carrying out the first method of the present invention is a method of designing a molecular structure of an inhibitor on HIV reverse transcriptase comprising the following steps (1) to (4):

(1) a step of determining, in the tertiary structure of rat DNA polymerase β, its pocket amino acid group consisting of plural amino acids to which nervonic acid is to bind. In the step (1), the plural amino acids are determined by comparing the ¹H-¹⁵N HMQC spectroscopy of rat DNA polymerase β in the absence of nervonic acid with the ¹H-¹⁵N HMQC spectroscopy of rat DNA polymerase β in the presence of nervonic acid, and selected from those having the absolute value of ¹H chemical shift of 0.06 ppm or more, and/or having the absolute value of ¹⁵N chemical shift of 0.4 ppm or more. In this example, the plural amino acids constituting the rat DNA polymerase β pocket amino acid group are determined to be Leu-11, Lys-35, His-51 and Thr-79.

(2) a step of determining the geometric configuration of the rat DNA polymerase β pocket amino acid group. In the step (2), the distances, in the tertiary structure of rat DNA polymerase β, between the Cα atoms of each of Leu-11, Lys-35, His-51 and Thr-79 constituting the rat DNA polymerase β pocket amino acid group are measured, thereby the geometric configuration of a triangular pyramid formed by Leu-11, Lys-35, His-51 and Thr-79 is determined.

(3) a step of detecting, in the tertiary structure of the amino acids of HIV reverse transcriptase, one or more candidates for a target pocket amino acid group having a geometric configuration similar to the geometric configuration of the rat DNA polymerase β pocket amino acid group determined in the step (2). In the step (3), amino acids constituting the candidate for the target pocket amino acid group are the same type as those constituting the rat DNA polymerase β pocket amino acid group, i.e., Leu, Lys, His and Thr. Further, the differences between (a) and (b) are within ±2×10⁻¹ nm, wherein (a) is respective distances, in the tertiary structure of HIV reverse transcriptase, between the Cα atoms of each of the amino acids constituting the target pocket amino acid group, and (b) is the respective distances between the Cα atoms obtained in the step (2).

(4) a step of determining by screening whether or not the candidate for a target pocket amino acid group is usable for designing the molecular structure of the inhibitor on HIV reverse transcriptase. In the step (4), the amino acids, Lys, Leu, His and Thr, which are selected as the amino acids constituting the candidate for the target pocket amino acid group are screened by the following two requirements:

(requirement 1) the amino acids are those present at the surface of HIV reverse transcriptase in the tertiary structure thereof; and

(requirement 2) the amino acids are those that have been conserved among species, when a comparison is made between the primary sequence of the amino acids of HIV reverse transcriptase and the primary sequence of the amino acids of immunodeficiency virus reverse transcriptase that is derived from an organism of a different species, in terms of biological classification, from human.

In this example, Lys-65, Leu-100, His-235 and Thr-386 of HIV reverse transcriptase satisfying the above two requirements were determined to be amino acids constituting the target pocket amino acid group usable for designing a molecular structure of the inhibitor on HIV reverse transcriptase.

In the example of carrying out the first method of the present invention, the molecular structure of the inhibitor on HIV reverse transcriptase is designed on the basis of the kinds of amino acid residues of rat DNA polymerase β, to which nervonic acid is to bind, and the geometric configuration of the amino acid residues.

In another aspect of the present invention, there is provided a method of designing a molecular structure of an inhibitor on an enzyme whose tertiary structures is unknown. Hereinafter, this method will be referred to as “the second method of the present invention”.

Specifically, the second method of the present invention is a method of designing a molecular structure of an inhibitor on a target enzyme whose tertiary structure is unknown, comprising:

analyzing, in the tertiary structure of a first enzyme whose primary, secondary and tertiary structures are known, a geometric configuration formed by amino acids to which a first inhibitor having biological, inhibitory activity on the first enzyme is to bind; and

searching, in a second enzyme whose primary, secondary and tertiary structures are known, amino acids constructing the similar geometric configuration to the geometric configuration that was analyzed in the first enzyme;

thereby designing the molecular structure of the inhibitor on the target enzyme whose tertiary structure is unknown.

In more detail, the second method of the present invention is a method of designing a molecular structure of an inhibitor on a target enzyme whose tertiary structure is unknown, comprising:

(1) a step of determining, in the tertiary structure of a first enzyme, a first pocket amino acid group consisting of plural amino acids to which a first inhibitor is to bind, wherein the plural amino acids are determined by comparing the ¹H-¹⁵N HMQC spectroscopy of the first enzyme in the absence of the first inhibitor with the ¹H-¹⁵N HMQC spectroscopy of the first enzyme in the presence of the first inhibitor, and selecting amino acids that satisfy the condition that the absolute value of ¹H chemical shift is equal to or more than a specific value and/or the absolute value of ¹⁵N chemical shift is equal to or more than another specific value;

(2) a step of determining the geometric configuration of the first pocket amino acid group, by measuring, in the tertiary structure of the first enzyme, the distances between the Cα atoms of each of the plural amino acids constituting the first pocket amino acid group;

(3) a step of detecting, in the tertiary structure of a second enzyme, a second pocket amino acid group having a geometric configuration similar to the geometric configuration formed by the first pocket amino acid group determined in the step (2), wherein it is presumed that the second pocket amino acid group has a geometric configuration similar to the geometric configuration formed by the first pocket amino acid group if the following requirements are satisfied:

(requirement 1) the second enzyme has the same biochemical activity as that of the target enzyme;

(requirement 2) the organism from which the second enzyme is derived, belongs to a different species, in terms of biological classification, from the organism from which the target enzyme is derived;

(requirement 3) the primary, secondary and tertiary structures of the second enzyme are known; and

(requirement 4) the second enzyme is biochemically inhibited by the first inhibitor, and

respective amino acids constituting the second pocket amino acid group are of the same kinds as the plural amino acids constituting the first pocket amino acid group determined in the step (2), and

the absolute values of differences between (a) respective distances, in the tertiary structure of the second enzyme, between the Cα atoms of each of the amino acids constituting the second pocket amino acid group, and (b) the respective distances between the Cα atoms in the first pocket amino acid group obtained in the step (2) are within a specific value; and

(4) a step of screening the amino acids constituting the second pocket amino acid group by the following two requirements:

(requirement 1) the amino acids are those present at the surface of the second enzyme in the tertiary structure thereof; and

(requirement 2) the amino acids are those that have been conserved among species, when a comparison is made among the primary sequence of the amino acids of the second enzyme, the primary sequence of the amino acid of the target enzyme and the primary sequence of the amino acids of another enzyme having the same biochemical activity as that of the second enzyme but derived from an organism of a different species from that of both the second enzyme and the target enzyme, in terms of biological classification; and

determining the amino acids satisfying the above two requirements to be those constituting the usable target pocket amino acid group for designing the molecular structure of the inhibitor on the target enzyme.

In the second method of the present invention, the molecular structure of the inhibitor on the target enzyme is designed on the basis of the kinds of amino acid residues of the first enzyme, to which the first inhibitor is to bind, and the geometric configuration of the amino acid residues.

An example of carrying out the second method of the present invention mentioned above is as follows. However, the second method of the present invention is not limited to this.

A method of designing a molecular structure of an inhibitor on human DNA topoisomerase II whose tertiary structure is unknown by analyzing, in the tertiary structure of rat DNA polymerase β, a geometric configuration formed by amino acids to which nervonic acid is to bind; searching, in yeast DNA topoisomerase II, the similar geometric configuration to the geometric configuration that was analyzed in rat DNA polymerase β; thereby designing the molecular structure of the inhibitor on human topoisomerase II on the basis of the kinds of amino acid residues of yeast DNA topoisomerase II, to which nervonic acid is to bind, and the geometric configuration of the amino acid residues.

In more detail, the example of carrying out the second method of the present invention is a method of designing a molecular structure of an inhibitor on human topoisomerase II comprising the following steps (1) to (4):

(1) a step of determining, in the tertiary structure of rat DNA polymerase β, a rat DNA polymerase β pocket amino acid group consisting of plural amino acids to which nervonic acid is to bind. In the step (1), the plural amino acids are determined by comparing the ¹H-¹⁵N HMQC spectroscopy of rat DNA polymerase β in the absence of nervonic acid with the ¹H-¹⁵N HMQC spectroscopy of rat DNA polymerase β in the presence of nervonic acid, and selected from those having the absolute value of ¹H chemical shift of 0.06 ppm or more, and/or having the absolute value of ¹⁵N chemical shift of 0.4 ppm or more. In this example, the plural amino acids constituting the rat DNA polymerase β pocket amino acid group are determined to be Leu-11, Lys-35, His-51 and Thr-79.

(2) a step of determining the geometric configuration of the rat DNA polymerase β pocket amino acid group. In the step (2), the distances, in the tertiary structure of rat DNA polymerase β, between the Cα atoms of each of Leu-11, Lys-35, His-51 and Thr-79 constituting the rat DNA polymerase β pocket amino acid group are measured, thereby the geometric configuration of a triangular pyramid formed by Leu-11, Lys-35, His-51 and Thr-79 is determined.

(3) a step of selecting, in the tertiary structure of yeast DNA topoisomerase II, a yeast DNA topoisomerase II pocket amino acid group consisting of Leu, Lys, His and Thr, and having a geometric configuration similar to the geometric configuration of the rat DNA polymerase β pocket amino acid group determined in the step (2). In the step (3), when the differences between (a) and (b) are within ±2×10⁻¹ nm, wherein (a) is respective distances, in the tertiary structure of the selected amino acids, between the Cα atoms of Leu, Lys, His and Thr constituting the yeast DNA topoisomerase II pocket amino acid group, and (b) is the respective distances between the Cα atoms obtained in the step (2), such Leu, Lys, His and Thr are determined to be the amino acids constituting the yeast DNA topoisomerase II pocket amino acid group.

(4) a step of determining by screening whether or not the yeast DNA topoisomerase II pocket amino acid group is usable for designing the molecular structure of the inhibitor on human DNA topoisomerase II. In the step (4), the amino acids, Leu, Lys, His and Thr, which are selected as the amino acids constituting the yeast DNA topoisomerase II pocket amino acid group are screened by the following two requirements:

(requirement 1) the amino acids are those present at the surface of yeast DNA topoisomerase II in the tertiary structure thereof; and

(requirement 2) the amino acids are those that have been conserved among species, when a comparison is made among the primary sequence of the amino acids of yeast DNA topoisomerase II, the primary sequence of the amino acids of human DNA topoisomerase II and the primary sequence of the amino acids of topoisomerase II derived from an organism of a different species from both yeast and human, in terms of biological classification; and

determining the amino acids, Thr-596, His-735, Leu-741 and Lys-983, satisfying the above two requirements to be usable for designing the molecular structure of the inhibitor on the target enzyme.

In the example of carrying out the second method of the present invention, the molecular structure of the inhibitor on human topoisomerase II is designed on the basis of the kinds of amino acid residues of yeast DNA topoisomerase II, to which nervonic acid is to bind, and the geometric configuration of the amino acid residues.

Another example of carrying out the second method of the present invention mentioned above is as follows. However, the second method of the present invention is not limited to this, either.

A method of designing a molecular structure of an inhibitor on human DNA topoisomerase II whose tertiary structure is unknown; by analyzing, in the tertiary structure of rat DNA polymerase β, a geometric configuration formed by amino acids to which lithocholic acid is to bind; searching, in yeast DNA topoisomerase II, the similar geometric configuration to the geometric configuration that was analyzed in rat DNA polymerase β; thereby designing the molecular structure of the inhibitor on human topoisomerase II on the basis of the kinds of amino acid residues of yeast DNA topoisomerase II, to which lithocholic acid is to bind, and the geometric configuration of the amino acid residues.

In more detail, the example of carrying out the second method of the present invention is a method of designing a molecular structure of an inhibitor on human topoisomerase II comprising the following steps (1) to (4):

(1) a step of determining, in the tertiary structure of rat DNA polymerase β, a rat DNA polymerase β pocket amino acid group consisting of plural amino acids to which lithocholic acid is to bind. In the step (1), the plural amino acids are determined by comparing the ¹H-¹⁵N HMQC spectroscopy of rat DNA polymerase β in the absence of lithocholic acid with the ¹H-¹⁵N HMQC spectroscopy of rat DNA polymerase β in the presence of lithocholic acid, and selected from those having the absolute value of ¹H chemical shift of 0.06 ppm or more, and/or having the absolute value of ¹⁵N chemical shift of 0.4 ppm or more. In this example, the plural amino acids constituting the rat DNA polymerase β pocket amino acid group are determined to be Lys-60, Leu-77 and Thr-79.

(2) a step of determining the geometric configuration of the rat DNA polymerase β pocket amino acid group. In the step (2), the distances, in the tertiary structure of rat DNA polymerase β, between the Cα atoms of each of Lys-60, Leu-77 and Thr-79 constituting the rat DNA polymerase β pocket amino acid group are measured, thereby the geometric configuration of a triangular shape formed by Lys-60, Leu-77 and Thr-79 is determined.

(3) a step of selecting, in the tertiary structure of yeast DNA topoisomerase II, a yeast DNA topoisomerase II pocket amino acid group consisting of Lys, Leu and Thr, and having a geometric configuration similar to the geometric configuration of the rat DNA polymerase β pocket amino acid group determined in the step (2). In the step (3), when the differences between (a) and (b) are within ±2×10⁻¹ nm, wherein (a) is respective distances, in the tertiary structure of the selected amino acids, between the Cα atoms of Lys, Leu and Thr constituting the yeast DNA topoisomerase II pocket amino acid group, and (b) is the respective distances between the Cα atoms obtained in the step (2), such Lys, Leu and Thr of the amino acids of rat polymerase β pocket amino acid group are determined to be the amino acids constituting the yeast DNA topoisomerase II pocket amino acid group.

(4) a step of determining by screening whether or not the yeast DNA topoisomerase II pocket amino acid group is usable for designing the molecular structure of the inhibitor on human DNA topoisomerase II. In the step (4), the amino acids, Lys, Leu and Thr, which are is selected as the amino acids constituting the yeast DNA topoisomerase II pocket amino acid group are screened by the following two requirements:

(requirement 1) the amino acids are those present at the surface of yeast DNA topoisomerase II in the tertiary structure thereof; and

(requirement 2) the amino acids are those that have been conserved among species, when a comparison is made among the primary sequence of the amino acids of yeast DNA topoisomerase II, the primary sequence of the amino acids of human DNA topoisomerase II and the primary sequence of the amino acids of topoisomerase II derived from an organism of a different species from both yeast and human, in terms of biological classification; and

determining the amino acids, Lys-720, Leu-760 and Thr-791, satisfying the above two requirements to be those constituting the target pocket amino acid group usable for designing the molecular structure of the inhibitor on the target enzyme.

In the example of carrying out the second method of the present invention, the molecular structure of the inhibitor on human topoisomerase II is designed on the basis of the kinds of amino acid residues of yeast DNA topoisomerase II, to which lithocholic acid is to bind, and the geometric configuration of the amino acid residues.

Now, the first method of the present invention, in which a molecular structure of an inhibitor on an enzyme whose secondary and tertiary structures are known will be described in more detail.

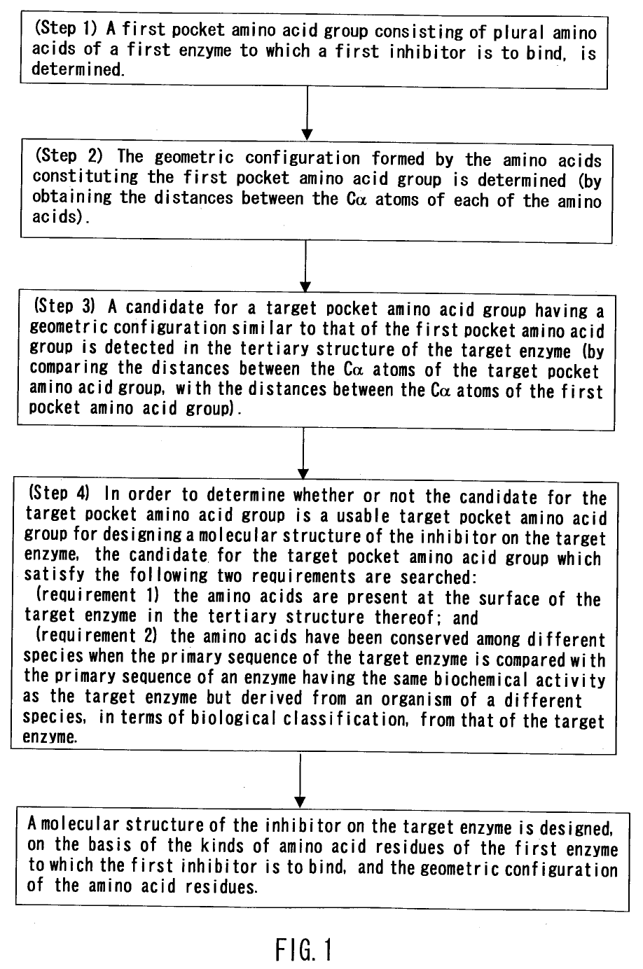

FIG. 1 is a block diagram showing the scheme of each process of the first method of the present invention.

The first method of the present invention is a method of designing a molecular structure of an inhibitor on a target enzyme whose secondary and tertiary structures are known, comprising; analyzing, in the tertiary structure of a first enzyme whose primary, secondary and tertiary structures are known, a geometric configuration formed by amino acids to which a first inhibitor having biological, inhibitory activity on the first enzyme is to be bound; and searching, in the target enzyme, amino acids constituting the similar geometric configuration to the geometric configuration that was analyzed in the first enzyme, thereby designing the molecular structure of the inhibitor on the target enzyme.

In the first method of the present invention, the primary, secondary and tertiary structures of the first enzyme used in the step (1) are known. Herein, the expression “the primary, secondary and tertiary structures are known” does not mean that every amino acid is in a state where assignment of its cross peak by NMR has been completed, but rather means that the primary, secondary and tertiary structures of the enzyme have been identified as a whole of the enzyme. In the present invention, it is convenient that using an enzyme whose primary, secondary and tertiary structures have already known. However, it is also possible to apply the first method of the present invention after analyzing the primary, secondary and tertiary structures of an enzyme. The primary, secondary and tertiary structures of an enzyme can be analyzed by employing the techniques known in the art, such as biochemical technique, NMR and X-ray crystal structure analysis, in combination.

One example of the enzyme whose primary, secondary and tertiary structures have been identified is rat DNA polymerase β (hereinafter also referred to as “rat pol. β”). Rat pol. β is the only enzyme whose primary, secondary and tertiary structures are known, among the DNA polymerase group which represent the enzymes that are essential to living organisms and catalyze the reaction of synthesizing DNA as the main body of genes.

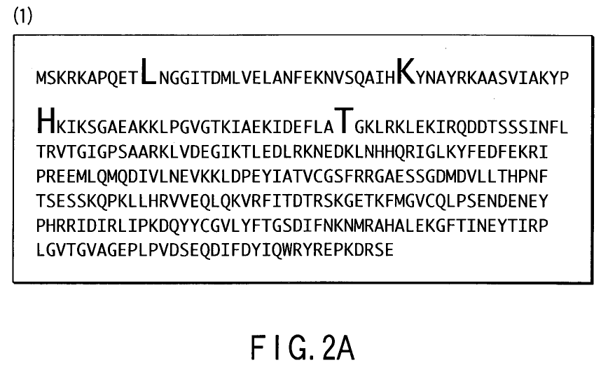

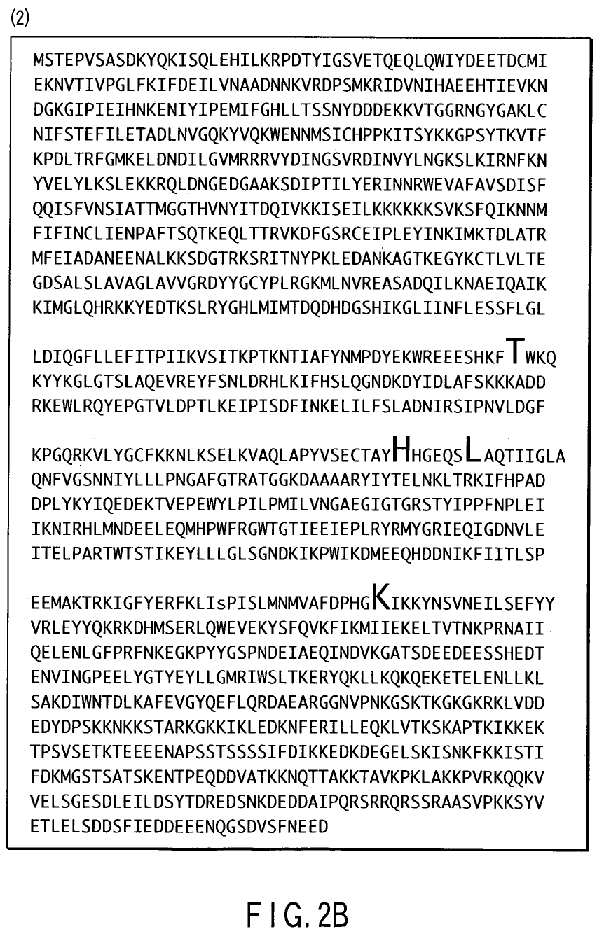

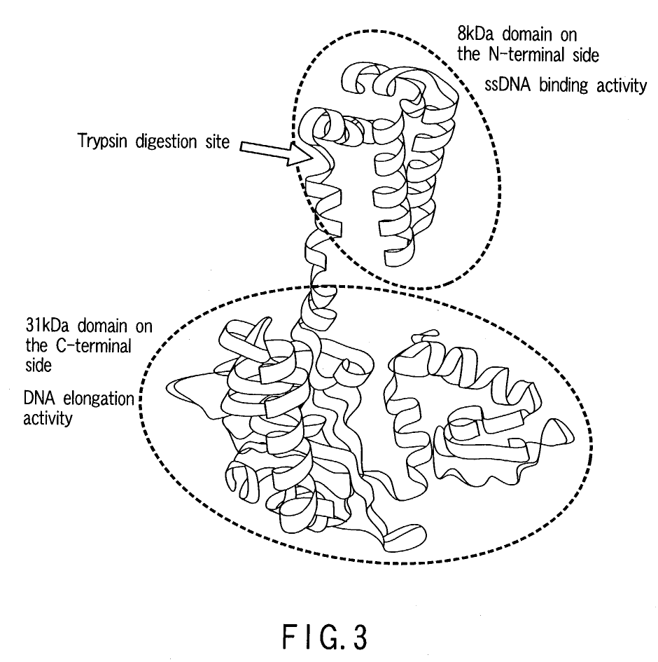

The amino acid sequence of rat pol. β is shown in reference 5, and the tertiary structure thereof (including the secondary structure) is shown in references 6 and 7. FIG. 2A shows the primary sequence of amino acid residues of rat pol. β (refer to the SQE ID No. 1 of the Sequence Listing described below). The amino acid sequence is available from SWISS-Plot Date Base. FIG. 3 is a schematic view showing the tertiary structure of rat pol. β, in which the principal chain of the amino acids is represented as a ribbon.

As shown in FIG. 3, rat pol. β is a single peptide constituted of the 8 kDa domain (1-87 amino acid residues) on the N-terminal side and the 31 kDa domain (88-335 amino acid residues) on the C-terminal side, in which the 31 kDa domain is connected with the 8 kDa domain. The 8 kDa domain can be cut off from the 31 kDa domain by mild hydrolysis with trypsin. Attribution of cross peaks by NMR has been completed in 75 residues of the total 87 amino acid residues of the 8 kDa domain (refer to reference 9).

The first inhibitor on the first enzyme used in the first method of the present invention is preferably an inhibitor whose inhibitory activity (e.g., IC₅₀) is in the order of μM or lower, although the first inhibitor is not particularly restricted thereto. The manner of inhibition may be either competition or non-competition. When the enzyme used in the first method of the present invention is rat pol. β, the first inhibitor is preferable to inhibit, in a competitive manner, the binding of rat pol. β to the DNA strand as the substrate.

Examples of the inhibitor having biochemical inhibitory activity on rat pol. β include nervonic acid (cis-15-tetracosenoic acid) and lithocholic acid (3-α-hydroxy-5β-cholanoic acid).

The chemical formulae of nervonic acid and lithocholic acid are shown below:

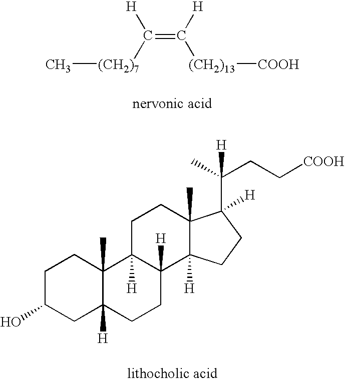

Nervonic acid is one of the fatty acids which most strongly inhibit rat pol. β. Lithocholic acid has been reported by Ogawa et al. (Jpn. J. Cancer Res. 89, 1154-1159, 1998), for example, as a representative example of steroids which are known as inhibitors on DNA polymerase. Both nervonic acid and lithocholic acid are commercially available and can easily be obtained by large amounts.

In the step (1) of the first method of the present invention, a first pocket amino acid group consisting of plural amino acids to which the first inhibitor is to bind, is determined in the tertiary structure of the first enzyme.

The plural amino acids constituting the first pocket amino acid group are decided by measuring the ¹H-¹⁵N HMQC (heteronuclear multiple-quantum correlated spectroscopy) of the first enzyme in the absence of the first inhibitor and the ¹H-¹⁵N HMQC spectroscopy of the first enzyme in the presence of the first inhibitor; overlapping the cross peak of each spectroscopy; and selecting amino acids that satisfy the condition that at least one of the absolute values of ¹H and ¹⁵N chemical shift of the amino acids is equal to or more than a specific value. Herein, the shift value is the one which can be set in accordance with the type or the like of the enzyme and the inhibitor used in the first method of the present invention. Although the values of ¹H and ¹⁵N chemical shift are generally set such that the number of the amino acid residues to be selected is three to four, the number of the amino acid residues is not restricted thereto. In the present invention, the number of amino acids is set in a range of three to four, because the minimum number of amino acids required to specify a geometric configuration formed by the amino acid residues is three and also the number of amino acids to be selected should not be uselessly enlarged in order to avoid making the identification task of the geometric configuration complicated.

The ¹H-¹⁵N HMQC spectroscopy can be measured by using Varian Unity-Plus 750 spectrometer, manufactured by Varian Co., Ltd. The measurement condition of the ¹H-¹⁵N HMQC spectroscopy may be set according to reference 4 listed below.

The amount of the inhibitor to be added when the ¹H-¹⁵N HMQC spectroscopy is measured in the presence of the inhibitor may be set by biochemically analyzing, in advance, the amount of the inhibitor which is required for inhibiting the enzymatic activity.

In the first method of the present invention, when rat pol. β and nervonic acid are used as the combination of the first enzyme and the fist inhibitor, respectively, the amount of nervonic acid to be added may be equimolar to the amount of rat pol. β. The absolute values of ¹H and ¹⁵N chemical shift as the indices may be set at 0.06 ppm or larger and 0.4 ppm or larger, respectively. In the amino acid sequence of rat pol. β, the amino acid residues that satisfy such ¹H or ¹⁵N chemical shift, are the following four amino acid residues: Leu-11, Lys-35, His-51 and Thr-79.

Further, in the first method of the present invention, when rat pol. βand lithocholic acid are used as the combination of the first enzyme and the fist inhibitor, respectively, the amount of lithocholic acid to be added may be equimolar to the amount of rat pol. β. The absolute values of ¹H and ¹⁵N chemical shift as the indices may be set at 0.06 ppm or larger and 0.6 ppm or larger, respectively. In the amino acid sequence of rat pol. β, the amino acid residues that satisfy such ¹H or ¹⁵N chemical shift are the following three amino acid residues: Lys-60, Leu-77 and Thr-79.

In the step (2) of the first method of the present invention, the geometric configuration of the first pocket amino acid group selected in the step (1) is determined. Specifically, the determination of the geometric configuration is carried out by obtaining, in the tertiary structure of the first enzyme, the distances between the Cα atoms of each of the amino acids constituting the first pocket amino acid group.

The distances between the Cα atoms can be calculated by using Protein Data Bank (Protein data Bank: 1BNO in the case of rat pol. β, and Protein data Bank: 1BJT in the case of yeast topo II), whereby the geometric configuration formed by the Cα atoms of the amino acids constituting the first pocket amino acid group can be analyzed. The technique of this analysis is described in detail in reference 4 listed below.

In the step (3) of the first method of the present invention, one or more candidates for a target pocket amino acid group having a geometric configuration similar to the geometric configuration of the rat DNA polymerase β pocket amino acid group determined in the step (2) is detected in the target enzyme. The detection is carried out by selecting the plural amino acids of the same kinds as the respective amino acids constituting the first pocket amino acid group determined in the step (2). Also, the distances, in the tertiary structure of the target enzyme, between the Cα atoms of each of the selected plural amino acids meet the following condition. That is, when comparison is made between (a) and (b), wherein (a) is the respective distances between the Cα atoms of each of the amino acids constituting the target pocket amino acid group, and (b) is the respective distances between the Cα atoms in the first pocket amino acid group obtained in the step (2), the differences in the corresponding distances between (a) and (b) are within a specific value.

Herein, “the corresponding distances between the Cα atoms of each of the amino acids” means that when the amino acids constituting the first pocket amino acid group are expressed as A, B and C and the amino acids constituting the target pocket amino acid group are expressed as a, b and c, the distance between the Cα atoms of A and B corresponds to the distance between the Cα atoms of a and b; the distance between the Cα atoms of B and C corresponds to the distance between the Cα atoms of b and c; and the distance between the Cα atoms of A and C corresponds to the distance between the Cα atoms of a and c. The amino acids A and a are the same kind to each other. Similarly, the amino acids B and b are the same kind, and the amino acids C and c are the same kind.

The differences in the corresponding distances between the Cα atoms of each of the amino acids may generally be set within a range of ±2×10⁻¹ nm, although the range may be changed in accordance with the type or the like of the first enzyme, the target enzyme and the first inhibitor used. It should be noted, however, that a too large difference in the corresponding distances between the Cα atoms of each of the amino acids represents a large difference between the geometric configuration formed by the amino acid residues constituting the first pocket amino acid group and that formed by the amino acid residues constituting the target pocket amino acid group, which is not preferable because the precision of the geometric configuration of the inhibitor on the target enzyme will then deteriorate.

In the first method of the present invention, when rat pol. β, nervonic acid and HIV reverse transcriptase are used as the first enzyme, the first inhibitor and the target enzyme, respectively, Leu, His, Thr and Lys having a geometric configuration similar to that of Leu-11, Lys-35, His-51 and Thr-79 of rat pol. β are Lys-65, Leu-100, His-235 and Thr-386 of HIV reverse transcriptase, which was decided as the candidate for a target pocket amino acid.

In the step (4) of the first method of the present invention, it is determined by searching whether or not the candidate for a target pocket amino acid group detected in the step (3) is usable for designing a molecular structure of an inhibitor on the target enzyme.

One feature of the first method of the present invention resides in this searching method. Specifically, the search is conducted on the basis of whether or not the amino acids constituting the candidate for a target pocket amino acid group determined in the step (3) satisfy the following two requirements:

(requirement 1) the amino acids are those present at the surface of the target enzyme in the tertiary structure thereof; and

(requirement 2) the amino acids are those that have been conserved among species, when a comparison is made between the primary sequence of the amino acids of the target enzyme and the primary sequence of the amino acids of another enzyme having the same biochemical activity as the target enzyme but derived from an organism of a different species from that of the target enzyme, in terms of biological classification.

When the amino acids of the candidate for a target pocket amino acid group determined in the step (3) satisfy the above two requirements, it is determined that the candidate(s) for a target pocket amino acid group is (are) usable for designing a molecular structure of an inhibitor on the target enzyme.

In requirement 1 of the step (4), it is required that the amino acids are those present at the surface of the target enzyme in the tertiary structure thereof, because it is presumed that such a pocket amino acid group must be present at the surface of the target enzyme in order for a specific substance (an inhibitor) to bind to the pocket amino acid group and inhibit the enzymatic activity of the target enzyme.

Further, in requirement 2 of the step (4), it is required that the amino acids are those that have been conserved among species, when a comparison is made between the primary sequence of the amino acids of the target enzyme and the primary sequence of the amino acids of another enzyme having the same biochemical activity as the target enzyme but derived from an organism of a different species from that of the target enzyme, in terms of biological classification. The reason thereof is that in a living organism, when amino acids which are essential for maintaining the activity of an enzyme (i.e., amino acids which are directly involved in the activity of the enzyme) are changed, the enzyme decreases its activity and, as a result, the living organism dies. That is, the requirement 2 is on the basis of an idea that the amino acids which play important roles in enzymatic activities will probably be maintained throughout the process of evolution of the organism.

Herein, the term “biological classification” represents genetic classification on the basis of the evolution theory of living organisms including virus. Accordingly, examples of “enzyme derived from an organism of a different species, in terms of biological classification” include enzymes derived from organisms belonging to different kingdoms, e.g., human (animal kingdom) vs. pea (plant kingdom), human (animal kingdom) vs. yeast (fungus kingdom), or enzymes derived from organisms of different species in the same animal kingdom (such as rat and mouse). In the case of virus, examples of “enzyme derived from an organism of a different species, in terms of biological classification” include enzymes derived from viruses whose hosts are of different species, in terms of biological classification (e.g., HIV vs. monkey immunodeficiency virus) and enzymes derived from viruses whose hosts are of the same species but whose genetic types are different (e.g., HIV-1 vs. HIV-2).

In the first method of the present invention, it is preferable that the search is carried out with respect to species which are as distant, in terms of both genetics and the evolution theory, as possible.

The technique which determines by searching whether or not any homology in amino acid sequence exists between a variety of different organisms has been practiced in the fields of molecular biology, information biology and the like. The present invention, however, has applied, for the first time, this technique in the field of molecular design.

It is preferable that plural types of enzyme are used in requirement 2 of the step (4), which enzymes are derived from an organism of a different species, in terms of biological classification, from the organism from which the target enzyme is derived. It is more preferable that at least ten enzymes from different species are searched. The larger the number of the enzymes, the higher precision is resulted in search, which is preferable.

With regards to the judgment on whether or not the amino acid is conserved in different species, the sample which exhibits homology of no less than 80%, in general, and preferably homology of 100%, is regarded as having “conserved amino acid”. It is preferable that as many amino acids as possible, of those constituting the pocket amino acid group, are the amino acids exhibiting 100% homology between the species. However, it is not necessary that all of the amino acids constituting the pocket amino acid group are the amino acids exhibiting 100% homology between the species. It suffices that at least one of the amino acids constituting the pocket amino acid group is the amino acid exhibiting 100% homology between the species.

Whether or not the amino acid satisfies the two requirements defined in the step (4) can be determined by using a computer software which is commercially available (INSIGHT II/BINDING SITE ANALYSIS, manufactured by MSI (Molecular Simulations Inc., U.S.A.)).

In the first method of the present invention, a molecular structure of a novel inhibitor on the target enzyme is designed, on the basis of the kinds of amino acid residues of the first enzyme, to which the first inhibitor is to bind, and the three-dimensional configuration of the amino acid residues.

Specifically, designing of a molecular structure of a novel inhibitor on the target enzyme is carried out by using the method including: specifying the substituent groups of the first inhibitor, which substituent groups are to bind to the amino acids constituting the first pocket amino acid group; adjusting the distances between each of the substituent groups and the three-dimensional configuration thereof such that these substituent groups are configured so that they fit in the geometric configuration formed by the first pocket amino acid group. The types of the substituent groups which are to bind to the amino acid residues constituting the first pocket amino acid group can be specified by using a biochemical method or the like which is known to those skilled in the art.

As described above, the first method of the present invention enables a molecular structure design of an inhibitor on an enzyme whose tertiary structure is determined and whose enzymatic activity is different from that of the first enzyme. Further, as a modification of the first method of the present invention, it is possible to design a molecular structure of another novel inhibitor on the first enzyme.

Specifically, the novel inhibitor on the first enzyme may be designed so that it has substituent groups that can be bound more firmly to the amino acids in the geometric configuration identified with the first enzyme in the steps (1) and (2) of the first method of the present invention and that accords the geometric configuration of the first enzyme.

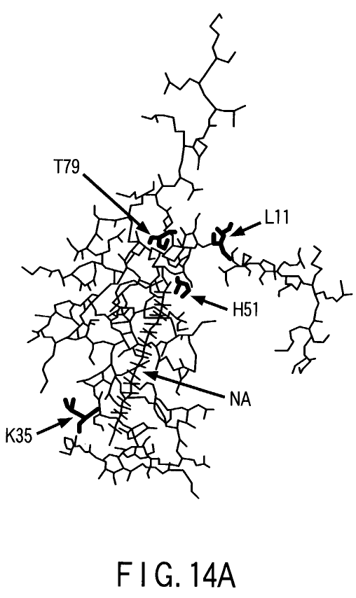

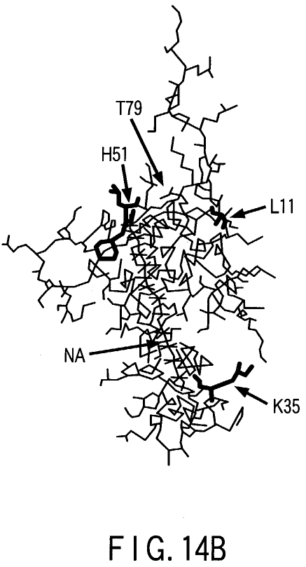

More specifically, in the binding between the amino acids, Leu-11, Lys-35, His-5 and Thr-79, of rat pol. β and nervonic acid, the carboxyl group at one terminal end of the nervonic acid chain is to bind to the hydrophilic site of Lys-35, and the methyl group at the other terminal end of the nervonic acid chain is to bind to Leu-11, His-51 and Thr-79, as shown in FIG. 14. The bent structure of the nervonic acid chain due to the double bond therein allows the nervonic acid chain to be fitted in the three-dimensional hydrophobic pocket formed by Leu-11, Lys-35, His-51 and Thr-79 of rat pol. β.

Accordingly, a molecular structure of a novel inhibitor other than nervonic acid, on rat pol. β, can be designed by adjusting the length of a fatty acid chain and the position of double bond so that the thus adjusted fatty acid is fitted in the three-dimensional pocket formed by Leu-11, Lys-35, His-51 and Thr-79 of rat pol. β in a more fitting manner.

Next, the second method of the present invention will be described in more detail.

The second method of the present invention is a method of designing a molecular structure of an inhibitor on a target enzyme whose tertiary structure is unknown, comprising: analyzing, in the tertiary structure of a first enzyme whose primary, secondary and tertiary structures are known, a geometric configuration formed by amino acids to which a first inhibitor having biological, inhibitory activity on the first enzyme is to be bound; and searching, in a second enzyme whose primary, secondary and tertiary structures are known, amino acids constructing the similar geometric configuration to the geometric configuration that was analyzed in the first enzyme; thereby designing the molecular structure of the inhibitor on the target enzyme.

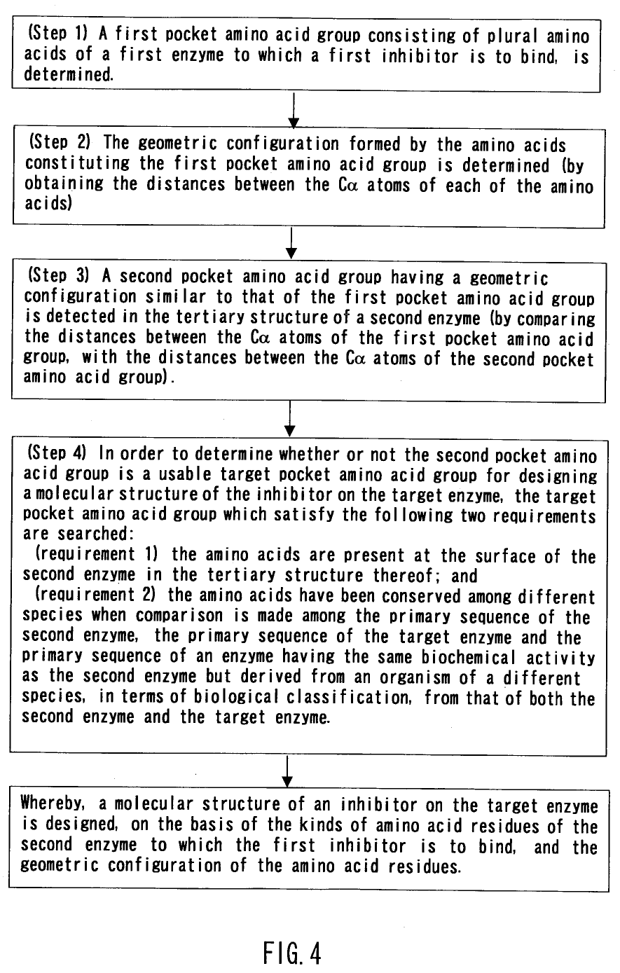

FIG. 4 is a block diagram showing the scheme of each process of the second method of the present invention.

The second method of the present invention is similar to the first method of the present invention, in that the second method employs basically the same technique utilized in the steps (1) to (4) of the latter.

That is, the first method of the present invention is to design a molecular structure of an inhibitor on a second enzyme, i.e., the target enzyme whose primary, secondary and tertiary structure have been identified, by utilizing the geometric configuration of a first enzyme. The second method of the present invention employs similar techniques on two types of enzymes, as in the first method, however, the second method is different from the first method in that the former then is to design a molecular structure of an inhibitor on a third enzyme, i.e., the target enzyme whose tertiary structure is unknown.

Herein, “a target enzyme” represents, as described above, an enzyme to which a molecular structure of an inhibitor is to be designed by implementing the methods of the present invention.

An enzyme generally has a relatively large molecular weight and a relatively complicated three-dimensional structure. Therefore, it is often extremely difficult in terms of technology to determine the tertiary structure, although the primary and secondary structures thereof can somehow be identified. Accordingly, in a group of enzymes that have the same biochemical activity but that exist in different organisms, there may be a case in which the tertiary structures of amino acids constituting the enzymes existing in some kinds of organism are known, while those of the enzymes existing in the rest of the organisms remain unknown. For example, DNA topoisomerase II, topo II, is an enzyme that enables breaking and reconnecting the double strand DNA, thereby eliminating twisting of DNA. Topo II is thus possessed by all the eucaryotes having a capacity of changing topology of DNA. Only the primary sequence of the amino acid of human topo II has been identified, although the amino acid sequence of topo II of yeast (S. cerevisiae) has been identified up to the tertiary structure level (reference 14 and reference 15 listed below). The second method of the present invention can be used for efficiently designing a molecular structure of an inhibitor on such an enzyme whose tertiary structure is unknown.

As described above, topo II is an enzyme capable of breaking and reconnecting the double strand DNA, thereby eliminating twisting of DNA and through which changing the topology of DNA. Since such a change in topology is essential in the development of cell division, the inhibitor on human topo II is likely to be applicable as an anticancer drug.

Next, the three types of enzymes, i.e., a first enzyme, a second enzyme and a target enzyme, employed in the second method of the present invention will be described in detailed below.

The structure of first and second enzymes have been identified up to the tertiary structural level. On the contrary, the target enzyme, at least the primary sequence of the amino acid thereof has been identified, but the tertiary structure thereof remains unknown. The first enzyme has biochemical activity of different type from that of the second and target enzymes. The second and target enzymes have biochemical activity of the same type, but they are derived from organisms of biologically different types to each other. The second method of the present invention can be used for designing a molecular structure of an inhibitor on an enzyme i.e., the target enzyme, which, due to the large molecular weight thereof, does not allow structural analysis by NMR.

A single type of inhibitor is employed against the enzyme used in the second method of the present invention. The inhibitor is capable of inhibiting the activity of the first enzyme. This inhibitor will be referred to as “the first inhibitor” hereinafter.

Specific examples of the combination of the first, second and target enzymes, and the first inhibitor, include the combination of rat pol. β, yeast topo II and human topo II, and nervonic acid; and the combination of rat pol. β, yeast topo II, and human topo II, and lithocholic acid.

In the second method of the present invention, the term “pocket amino acid group” is used for each of the first, second and target enzymes. As described below, plural amino acids, in the enzyme, satisfying a specific condition, is allocated to each of the pocket amino acid groups.

In the first enzyme used in the step (1) of the second method of the present invention, the primary, the secondary and the tertiary structures are known. Herein, the expression “the primary, secondary and tertiary structures are known” does not mean that every amino acid is in a state where assignment of its cross peak by NMR has been completed, but rather means that the primary, secondary and tertiary structures of the enzyme have been identified as a whole of the enzyme.

In the present invention, it is convenient that using an enzyme whose primary, secondary and tertiary structures have already known. However, it is also possible to apply the second method of the present invention after analyzing the primary, secondary and tertiary structures of an enzyme. The primary, secondary and tertiary structures of an enzyme can be analyzed by employing the techniques known in the art, such as biochemical technique, NMR and X-ray crystal structure analysis, in combination.

One example of the enzyme whose primary, secondary and tertiary structures have been identified is rat pol. β. It is the only one enzyme whose primary, secondary and tertiary structures are known, among the DNA polymerase group which represent the enzymes that are essential to living organisms and catalyze the reaction of synthesizing DNA as the main body of genes.

The first inhibitor on the first enzyme used in the second method of the present invention is preferably an inhibitor whose inhibitory activity (e.g., IC₅₀) is in the order of μM or lower, although the first inhibitor is not particularly restricted thereto. The manner of inhibition may be either competition or non-competition. When the enzyme used in the second method of the present invention is rat pol. β, the first inhibitor is preferable to inhibit, in a competitive manner, the binding of rat pol. β to the DNA strand as the substrate.

Examples of the inhibitor having biochemical inhibitory activity on rat pol. β include nervonic acid and lithocholic acid.

In the step (1) of the second method of the present invention, a first pocket amino acid group consisting of plural amino acids to which the first inhibitor is to bind, is determined in the tertiary structure of the first enzyme.

The plural amino acids constituting the first pocket amino acid group are decided by measuring the ¹H-¹⁵N HMQC of the first enzyme in the absence of the first inhibitor and the ¹H-¹⁵N HMQC spectroscopy of the first enzyme in the presence of the first inhibitor; overlapping the cross peak of each spectroscopy; and selecting amino acids that satisfy the condition that at least one of the absolute values of ¹H and ¹⁵N chemical shift of the amino acids is equal to or more than a specific value. Herein, the shift value is the one which can be set in accordance with the type or the like of the enzyme and the inhibitor used in the second method of the present invention. Although the values of ¹H and ¹⁵N chemical shift are generally set such that the number of the amino acid residues to be selected is three to four, the number of the amino acid residues is not restricted thereto. In the present invention, the number of amino acids is set in a range of three to four, because the minimum number of amino acids required to specify a geometric configuration formed by the amino acid residues is three and also the number of amino acids to be selected should not be uselessly enlarged in order to avoid making the identification task of the geometric configuration complicated.

The ¹H-¹⁵N HMQC spectroscopy can be measured by using Varian Unity-Plus 750 spectrometer, manufactured by Varian Co., Ltd. The measurement condition of the ¹H-¹⁵N HMQC spectroscopy may be set according to reference 4 listed below.

The amount of the inhibitor to be added when the ¹H-¹⁵N HMQC spectroscopy is measured in the presence of the inhibitor may be set by biochemically analyzing, in advance, the amount of the inhibitor which is required for inhibiting the enzymatic activity.

In the second method of the present invention, when rat pol. β and nervonic acid are used as the combination of the first enzyme and the fist inhibitor, respectively, the amount of nervonic acid to be added may be equimolar to the amount of rat pol. β. The absolute values of ¹H and ¹⁵N chemical shift as the indices may be set at 0.06 ppm or larger and 0.4 ppm or larger, respectively. In the amino acid sequence of rat pol. β, the amino acid residues that satisfy such ¹H or ¹⁵N chemical shift, are the following four amino acid residues: Leu-11, Lys-35, His-51 and Thr-79.

Further, in the second method of the present invention, when rat pol. β and lithocholic acid are used as the combination of the first enzyme and the fist inhibitor, respectively, the amount of lithocholic acid to be added may be equimolar to the amount of rat pol. β. The absolute values of ¹H and ¹⁵N chemical shift as the indices may be set at 0.06 ppm or larger and 0.6 ppm or larger, respectively. In the amino acid sequence of rat pol. β, the amino acid residues that satisfy such ¹H or ¹⁵N chemical shift are the following three amino acid residues: Lys-60, Leu-77 and Thr-79.

In the step (2) of the second method of the present invention, the geometric configuration of the first pocket amino acid group selected in the step (1) is determined. Specifically, the determination of the geometric configuration is carried out by obtaining, in the tertiary structure of the first enzyme, the distances between the Cα atoms of each of the amino acids constituting the first pocket amino acid group.

The distances between the Cα atoms can be calculated by using Protein Data Bank (Protein data Bank: 1BNO in the case of rat pol. β, and Protein data Bank: 1BJT in the case of yeast topo II), whereby the geometric configuration formed by the Cα atoms of the amino acids constituting the first pocket amino acid group can be analyzed. The technique of this analysis is described in detail in reference 4 listed below.

In the step (3) of the second method of the present invention, a second pocket amino acid group having a geometric configuration similar to the geometric configuration of the first pocket amino acid group identified in the step (2) is detected in the second enzyme. The second enzyme used in the step (3) satisfies the following requirements:

(requirement 1) the second enzyme has the same biochemical activity as the target enzyme;

(requirement 2) the organism from which the second enzyme is derived, belongs to a different type, in terms of biological classification, from the organism from which the target enzyme is derived;

(requirement 3) the primary, secondary and tertiary structures of the second enzyme are known; and

(requirement 4) the second enzyme is biochemically inhibited by the first inhibitor.

The expression that “(an enzyme) has the same biochemical activity as the target enzyme” defined in requirement 1 of the step (3) means that the enzyme catalyzes, in the enzymatic reaction thereof, substantially the same substrate as the target enzyme does, thereby generating substantially the same product in the biochemical reaction.

The expression that “the organism from which the second enzyme is derived belongs to a different type, in terms of biological classification, from the organism from which the target enzyme is derived” defined in requirement 2 of the step (3) represents the same meaning as described in the first method. Further, as is in the first method, it is preferable that the organism from which the second enzyme is derived, is as distant as possible, in terms of biological classification, from the organism from which the target enzyme is derived.

With regards to “the primary, secondary and tertiary structure of an enzyme” defined in requirement 3 of the step (3), it is convenient that an enzyme whose primary, secondary and tertiary structures are already known is used, as described above. However, it is possible to apply the second method of the present invention after analyzing the primary, secondary and tertiary structures of an enzyme. The primary, secondary and tertiary structures of an enzyme can be analyzed by employing the techniques known in the art, such as biochemical technique, NMR and X-ray crystallographic analysis, in combination.

The expression that “(the second enzyme) is biochemically inhibited by the first inhibitor” defined in requirement 4 of the step (3) represents that the value of the inhibitory activity IC₅₀ of the first inhibitor effected against the second enzyme is preferably in a range 10 times to 1/10 times as much as, and more preferably substantially the same as the value of the inhibitory activity IC₅₀ of the first inhibitor against the first enzyme.

In the step (3) of the second method of the present invention, a second pocket amino acid group having a geometric configuration similar to that of the first pocket amino acid group is detected in the second enzyme. The detection is carried out by selecting the plural amino acids of the same kinds as the respective amino acids constituting the first pocket amino acid group determined in the step (2). Also, the distances, in the tertiary structure of the second enzyme, between the Cα atoms of each of the selected plural amino acids meet the following condition. That is, when comparison is made between (a) and (b), wherein (a) is the respective distances between the Cα atoms of each of the amino acids constituting the second pocket amino acid group, and (b) is the respective distances between the Cα atoms in the first pocket amino acid group obtained in the step (2), the differences between (a) and (b), i.e., the corresponding distances between the Cα atoms of each of the amino acids, are within a specific value.

Herein, “the corresponding distances between the Cα atoms of each of the amino acids” has the same meaning as described in the first method of the present invention.

The corresponding distances between the Cα atoms of each of the amino acids may generally be set within a range of ±2×10⁻¹ nm, although the range may be changed in accordance with the type or the like of the first enzyme, the second enzyme and the first inhibitor used. It should be noted, however, if the large distance may be set, it makes difference large between the geometric configurations of the first pocket amino acid group and the second pocket amino acid group. It is not preferable because the precision of the geometric configuration of the inhibitor on the target enzyme will then deteriorate.

When rat pol. β, yeast topo II, human topo II and nervonic acid are used as the first, second, target enzymes and the first inhibitor, respectively, the specific value may be set within a range of ±2×10⁻¹ nm. Similarly, the specific value may be set within the same range when lithocholic acid is used as the first inhibitor.

In the step (4) of the second method of the present invention, it is determined by searching whether or not the second pocket amino acid group determined in the step (3) is usable for designing a molecular structure of an inhibitor of the target enzyme.

One of the features of the second method of the present invention resides in the searching method. Specifically, the search is conducted on the basis of whether or not the second pocket amino acid group satisfies the following two requirements:

(requirement 1) the amino acids are those present at the surface of the target enzyme in the tertiary structure thereof; and

(requirement 2) the amino acids are those that have been conserved among species, when a comparison is made among the primary sequence of the amino acids of the second enzyme, the primary sequence of the amino acids of the target enzyme and the primary sequence of the amino acids of another enzyme having the same biochemical activity as the second enzyme but derived from an organism of a different species from that of both the second and target enzymes, in terms of biological classification.

When the amino acids of the second pocket amino acid group satisfy the two requirements, it is determined that the second pocket amino acid group are usable for designing a molecular structure of an inhibitor on the target enzyme.

The reason why the two requirements are employed in the step (4) is the same as that described in the first method.

With regards to the number of the enzyme derived from different species, in terms of biological classification used in requirement 2 of the step (4), at least two enzymes (preferably at least ten enzymes) are searched. The larger the number of the enzyme, the higher precision is resulted in search, which is preferable.

With regards to the judgment on whether or not the amino acid is conserved in different species, the sample which exhibits homology of no less than 80%, in general, and preferably homology of 100%, is regarded as “conserved amino acid”. It is preferable that as many amino acids as possible, of those constituting the pocket amino acid group, are the amino acids exhibiting 100% homology between the species. However, it is not necessary that all of the amino acids constituting the pocket amino acid group are the amino acids exhibiting 100% homology between the species. It suffices that one of the amino acids constituting the pocket amino acid group is the amino acid exhibiting 100% homology between the species.

Whether or not the amino acid residues constituting the second pocket amino acid group determined in the step (3) has a three-dimensional configuration similar to the geometric configuration of the first pocket amino acid group, and whether or not the amino acid residues constituting the second pocket amino acid group satisfy the two requirements defined in the step (4), can be determined by using a computer software which is commercially available (INSIGHT II/BINDING SITE ANALYSIS, manufactured by MSI (Molecular Simulations Inc.)).

When rat pol. β, yeast topo II and human topo II, and nervonic acid are used as the first, second and target enzymes, and the first inhibitor, Thr-596, His-735, Leu-741 and Lys-983 among the amino acid residues constituting the second pocket amino acid group of yeast topo II satisfy each of the requirements in the step (3) and the step (4). On the other hand, when lithocholic acid is used as the first inhibitor, Lys-720, Leu-760 and Thr-791 satisfy these requirements.

In the second method of the present invention, when the second pocket amino acid group of the second enzyme satisfies the two requirements defined in the step (4), it is presumed that the target pocket amino acid group is present in the target enzyme. Specifically, it is presumed that respective amino acid residues constituting the target pocket amino acid group are of the same kinds as those constituting the second pocket amino acid group, and the amino acid residues of the target pocket amino acid group are arranged in a geometric configuration similar to that of the second amino acid group.

In the second method of the present invention, after the presumption as described above is carried out, a molecular structure of an inhibitor on the target enzyme is designed on the basis of the kinds of amino acid residues of the second enzyme, to which the first inhibitor is to bind, and the geometric configuration of the amino acid residues.

With regards to a specific designing method, that described in the first method can be employed.

Next, examples of the present invention will be described hereinafter. It should be noted that the present invention is not restricted to these examples.

### EXAMPLE 1

In this example, rat pol. β, nervonic acid and HIV reverse transcriptase were used as the first enzyme, the first inhibitor and the target enzyme, respectively, for designing a molecular structure of an inhibitor on HIV reverse transcriptase.

<1-1> The primary sequence of the amino acid residue of rat pol. β is shown in FIG. 2A. The model of the tertiary structure of rat pol. β is shown in FIG. 3.

<1-2> Measurement of Inhibitory Activity Effected by Nervonic acid on Rat Pol. β

The activity of rat pol. β was measured according to the method described in reference 3 listed below, by using poly(dA)/oligo(dT)₁₂₋₁₈ (template DNA) and [³H]-dTTP (nucleotide) as substrates and measuring radiation of [³H]-dTTP incorporated in the presence of nervonic acid of different concentrations.

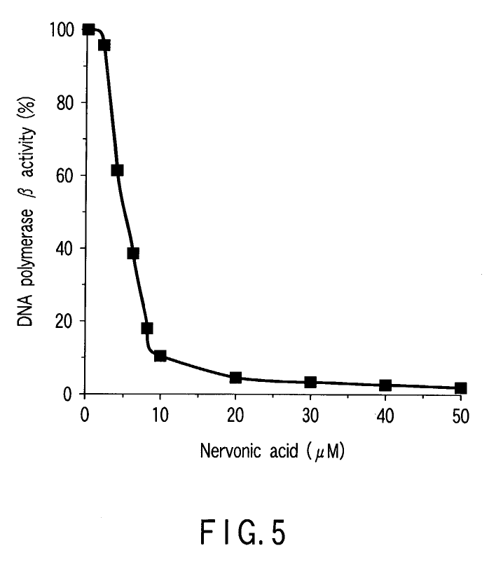

FIG. 5 is a graph showing an inhibition curve. In FIG. 5, the vertical axis represents the activity of rat pol. β and the horizontal axis represents the concentration of nervonic acid.

<1-3> Measurements of ¹H-¹⁵N HMQC Spectroscopy of Rat Pol. β in the Presence and the Absence of Nervonic Acid

A recombinant E. coli having 8 kDa domain of rat pol. β was constructed according to the method described in reference 4 listed below. ¹⁵NH₄Cl as the nitrogen source was added to culture medium of the recombinant E. coli for labeling. In the absence and the presence (1.25 mM, which is equimolar to 8 kDa domain of rat pol. β) of nervonic acid, ¹H-¹⁵N HMQC spectroscopy (750 Mz) was measured by using Varian Unity-Plus 750 spectrometer, manufactured by Varian Co., Ltd. The experiment was conducted only with 8 kDa domain of rat pol. β, because it has been confirmed that the amino acid residues to which nervonic acid is to bind are present only at the 8 kDa domain (references 3 and 4 listed below).

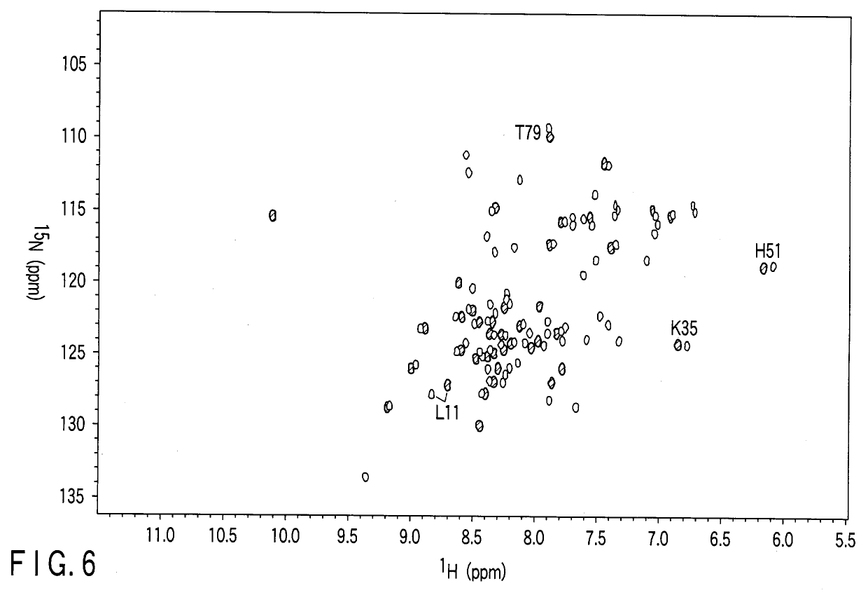

FIG. 6 shows the cross peaks of ¹H-¹⁵N HMQC spectroscopy of rat pol. β. In FIG. 6, the vertical axis represents the shift value of ¹⁵N and the horizontal axis represents the shift value of ¹H. FIG. 6 shows the cross peaks of amino acid residues whose ¹H and ¹⁵N chemical shifts were 0.015 ppm or more and 0.1 ppm or more, respectively, in a manner that the graph of the cross peaks in the presence of nervonic acid and the graph of the cross peaks in the absence of nervonic acid are overlapped. In FIG. 6, among the amino acid residues, those in which the absolute values of ¹H and ¹⁵N chemical shift were 0.06 ppm or more and 0.4 ppm or more, respectively, i.e., four amino acid residues including Leu-11, Lys-35, His-51 and Thr-79, are each indicated by one alphabetic letter representing the type of the amino acid and the sequence number thereof. The group consisting of these four amino acid residues will be referred to as “rat pol. βpocket amino acid group-1” hereinafter.

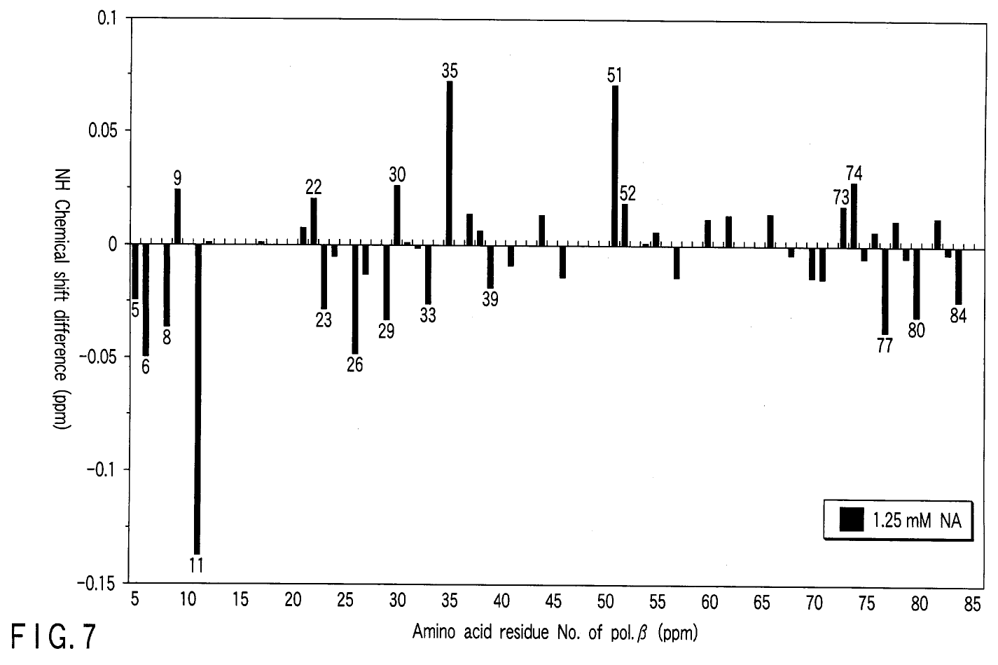

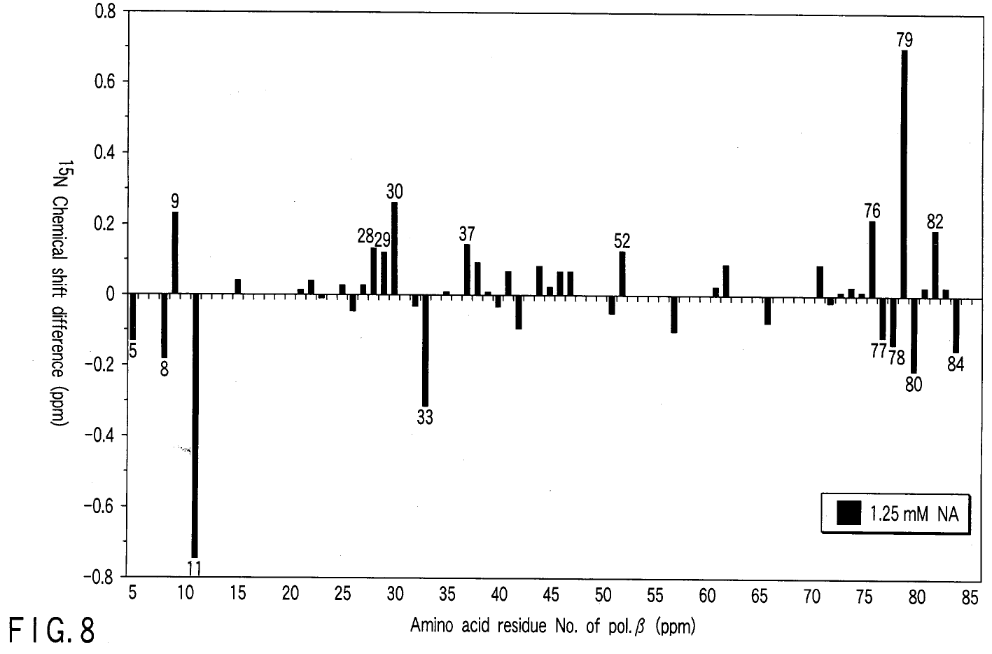

FIG. 7 is a bar graph in which the cross peaks of ¹H-¹⁵N HMQC spectroscopy shown in FIG. 6 is expressed as the relationship of ¹H chemical shift value vs. the amino acid residue number. FIG. 8 is a bar graph in which the cross peaks of ¹H-¹⁵N HMQC spectroscopy shown in FIG. 6 is expressed as the relationship of ¹⁵N chemical shift value vs. the amino acid residue number. In FIGS. 7 and 8, the vertical axis represents the chemical shift value and the horizontal axis represents the sequence number of the amino acid.

<1-4> Measurement of the Distances Between the Cα Atoms of the Rat Pol. β Pocket amino Acid Group-1

The distances between the Cα atoms of each of Leu-11, Lys-35, His-51 and Thr-79 constituting the rat pol. β pocket amino acid group-1 obtained in the <1-3> were measured by using Protein Data Bank; 1BNO and with reference to references 14, 15 and 16 listed below. The thus obtained distances between the Cα atoms are shown in Table 1.

TABLE 1

| Distance between | |
|---|---|
| the Cα atoms of the | |
| amino acid residues | Distance (×10⁻¹ nm) |
| Leu-11 and His-51 | 10.41 |
| Leu-11 and Thr-79 | 12.79 |
| Leu-11 and Lys-35 | 27.95 |
| His-51 and Thr-79 | 15.95 |
| His-51 and Lys-35 | 21.87 |
| Thy-79 and Lys-35 | 24.88 |

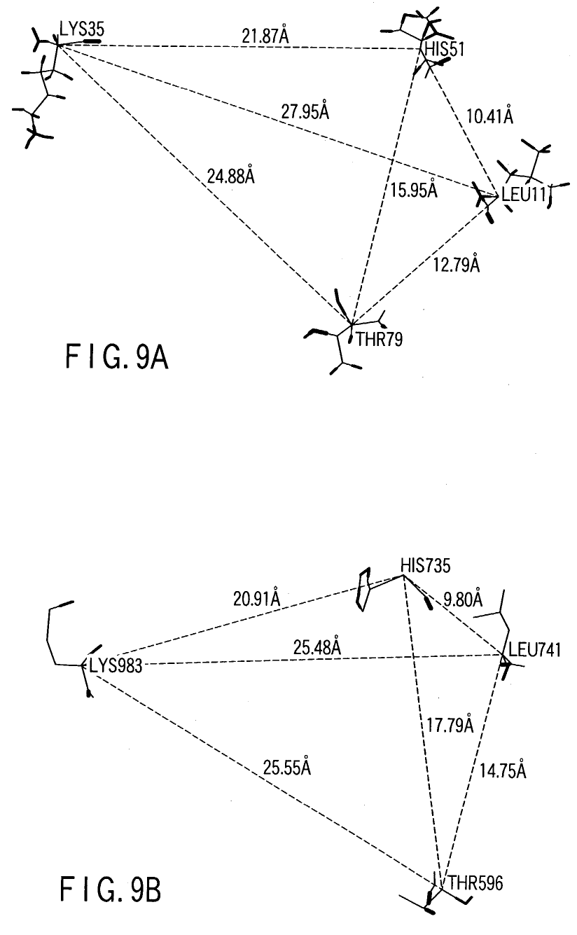

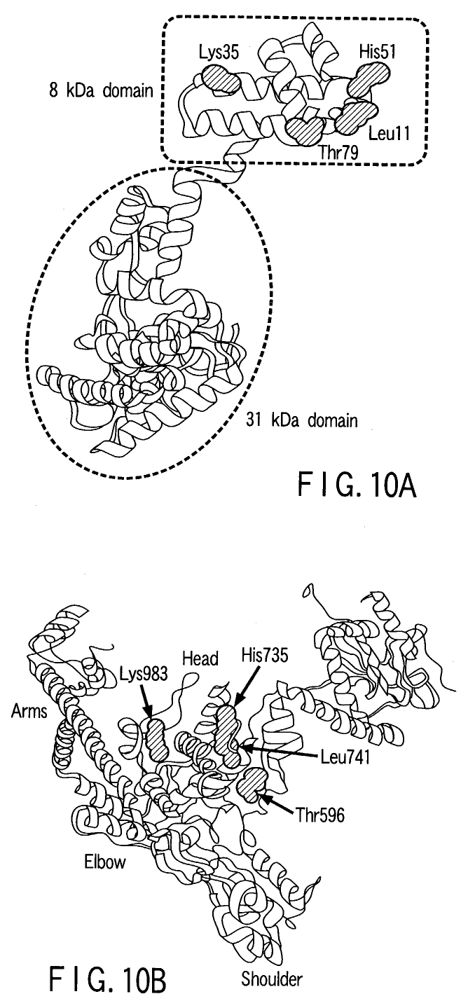

FIG. 9A is a view showing, in three dimensional manner, the geometric configuration of the Cα atoms of the amino acid residues distanced from each other as shown in Table 1 (a computer software (INSIGHT II/BINDING SITE ANALYSIS, manufactured by Molecular Simulations Inc., U.S.A) was employed). Further, FIG. 10A is a view of a model in which the four amino acid residues constituting the rat pol. β pocket amino acid group-1, to which nervonic acid has bound, are indicated in rat pol. β represented as a ribbon.

<1-5> Determination, by Searching, of a Target Pocket Amino Acid Group on HIV Reverse Transcriptase

The target pocket amino acid group on HIV reverse transcriptase was searched by the following items 1) to 3) using a computer software (INSIGHT II/BINDING SITE ANALYSIS, manufactured by Molecular Simulations Inc., U.S.A).

Item 1) determination, by searching, of whether or not the target pocket amino acid group (HIV pocket amino acid group-1) corresponding to the rat pol. β pocket amino acid group-1 is present in the amino acid sequence of HIV reverse transcriptase;

Item 2) determination, by searching, of whether or not the amino acids constituting the HIV pocket amino acid group-i obtained in the above item 1) are present at the surface of HIV reverse transcriptase in the tertiary structure thereof; and

Item 3) determination, by searching, of whether or not the amino acids constituting the HIV pocket amino acid group-1 are the amino acids conserved in immunodeficiency virus reverse transcriptases between other types which derived from different organisms, in terms of biological classification.

The distances between each of the Cα atoms obtained above are shown in Table 2.

TABLE 2

| Distance between | |
|---|---|
| the Cα atoms of the | |
| amino acid residues | Distance (×10⁻¹ nm) |
| Leu-100 and His-235 | 10.94 |
| Leu-100 and Thr-386 | 16.02 |
| Leu-100 and Lys-65 | 25.22 |
| His-235 and Thr-386 | 19.24 |
| His-235 and Lys-65 | 20.86 |
| Thy-386 and Lys-65 | 21.39 |

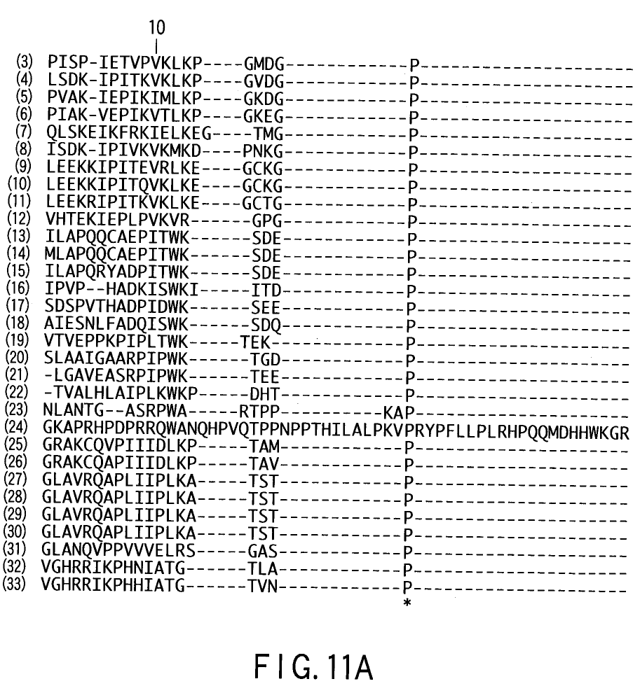

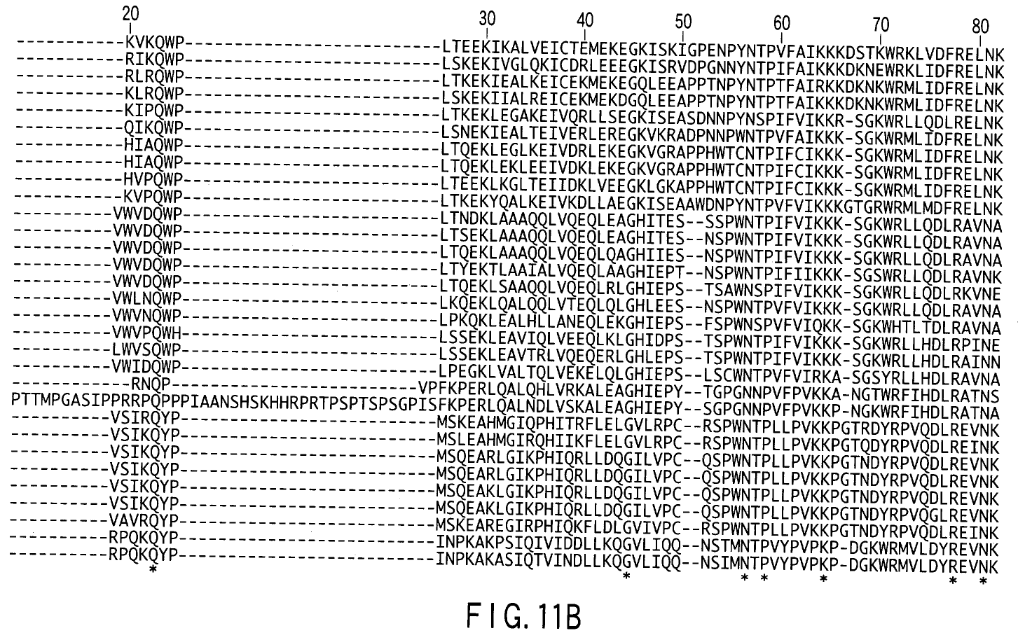

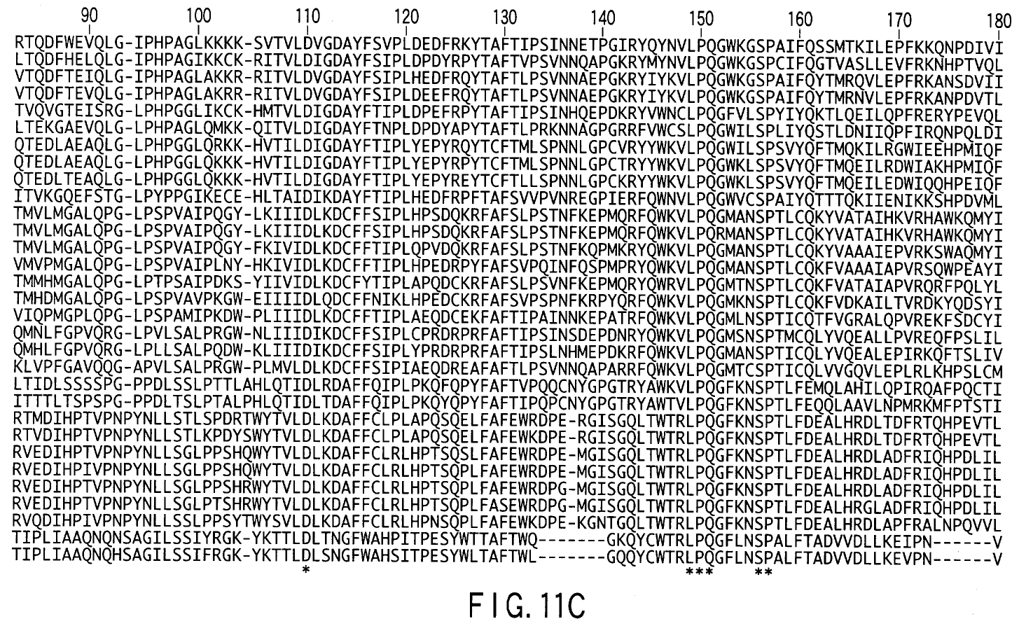

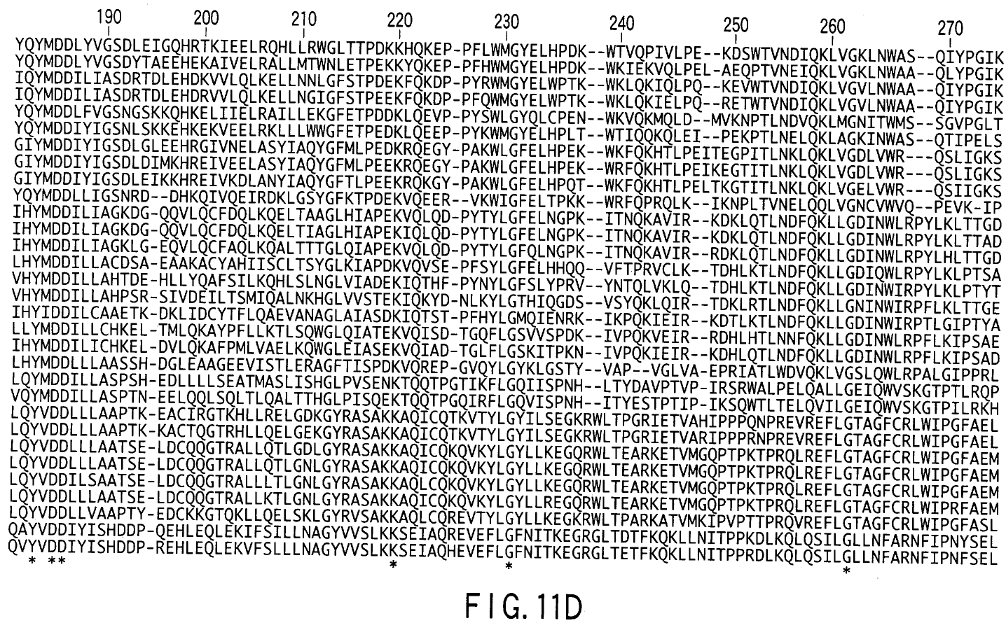

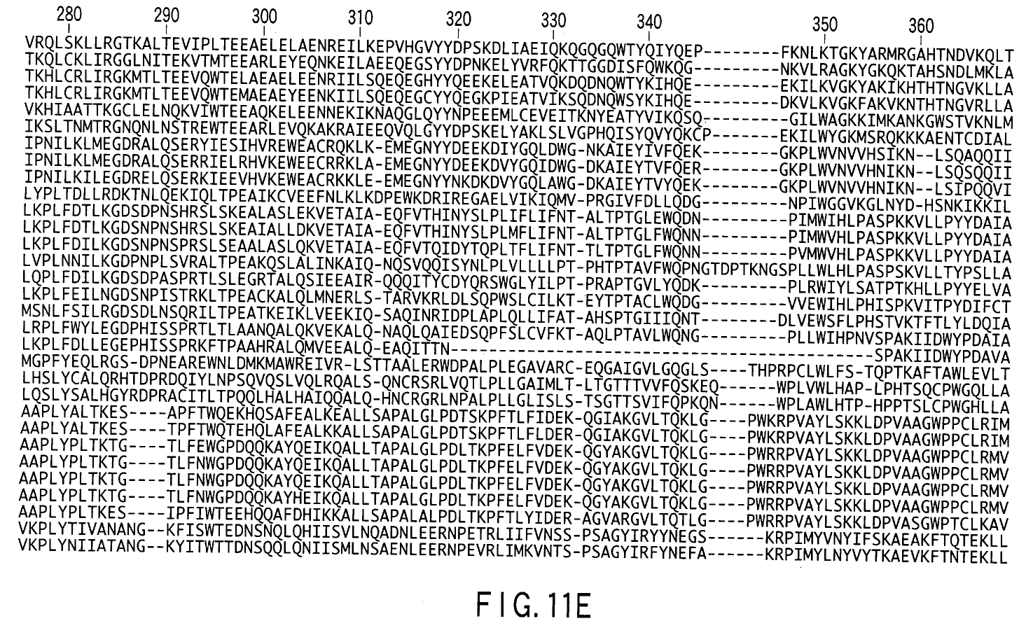

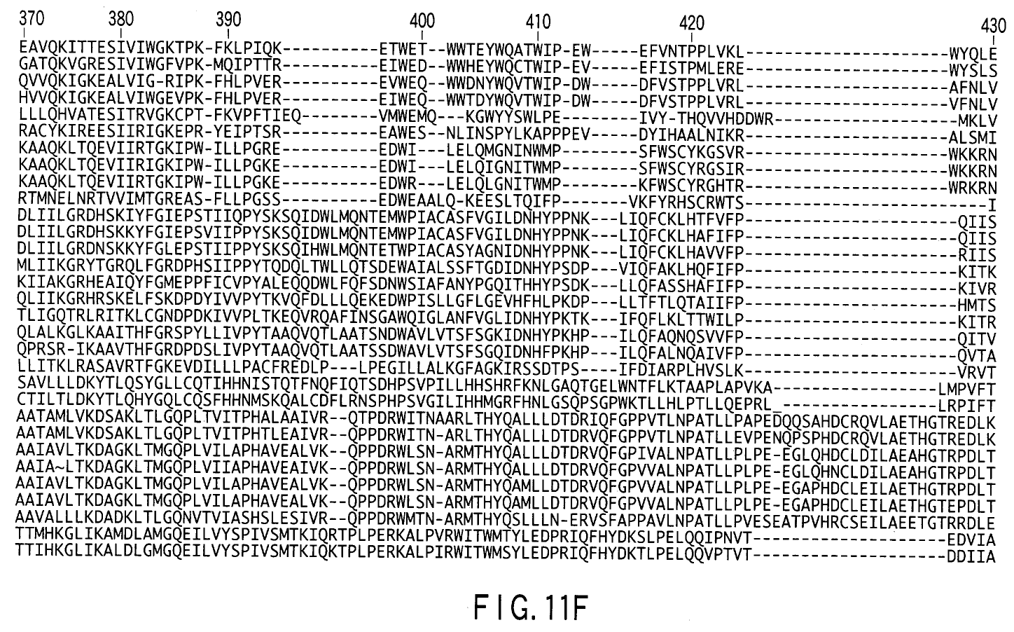

In the searching of the above item 1), the amino acids of HIV reverse transcriptase having such distances between each of the Cα atoms as within a range of ±2×10⁻¹ nm with respect to the corresponding distances between each of the Cα atoms in pol. β shown in Table 1, were selected. Further, in the searching of the above items 2) and 3), the 31 sequences of the immunodeficiency virus reverse transcriptase each derived from organisms of different species shown in FIGS. 11A-11F were used.

FIGS. 11A-11F shows the 31 sequences of the immunodeficiency virus reverse transcriptase, which correspond to SQE ID Nos. 3 to 33, respectively, of the Sequence Listing shown below. Note that only the amino acids up to No. 100 are shown in the Sequence Listing. Each of the amino acid sequences is available from SWISS-Plot Data Base. In the sequences of FIGS. 11A-11F, a hyphen(s) is (are) inserted between specific amino acids so that conservation of the amino acid sequences among any reverse transcriptases can easily be detected (the INSIGHT II/BINDING SITE ANALYSIS was used).

As shown in FIG. 11B, the Lys (K) as the amino acid No. 65 is conserved throughout the 31 enzymes searched. With regards to the other amino acids (e.g., the amino acid No. 100 shown in FIG. 11C) are not always conserved throughout the 31 enzymes. However, even if the amino acid is not completely conserved throughout the searched sequences, the amino acid which satisfies the items 1) and 2) mentioned above can be regarded as the amino acid constituting the target amino acid group. Herein, it is an essential requirement that the types of the amino acids constituting the rat pol. β pocket amino acid group-1 are identical with the types of the amino acids constituting HIV pocket amino acid group-1. Although the other requirements (i.e., the distances between the Cα atoms of each of the amino acids, the amino acids being present at the surface of the enzyme, and the amino acids being conserved among different organisms in terms of biological classification) are not necessarily essential, there is a decreasing priority in the order of mentioning in determining the target amino acid group, i.e., the requirement of the distances between the Cα atoms is given a highest priority.

As a result of the determination by searching, it has been confirmed that, in HIV reverse transcriptase, Lys-65, Leu-100, His-235 and Thr-386 constituting HIV pocket amino acid group-1 satisfy the above items 1), 2) and 3).

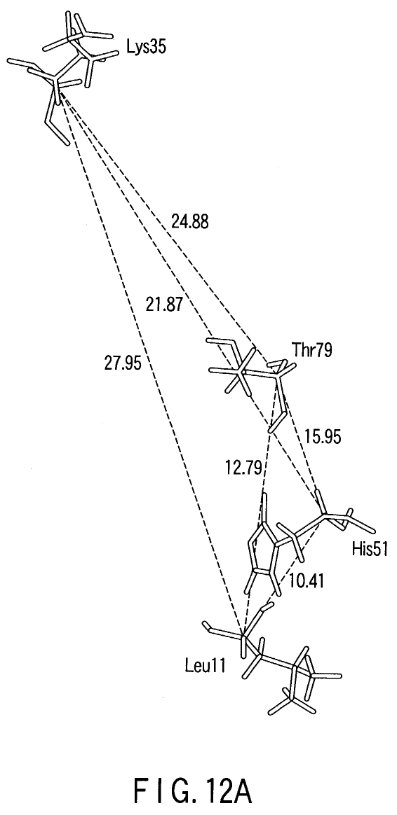

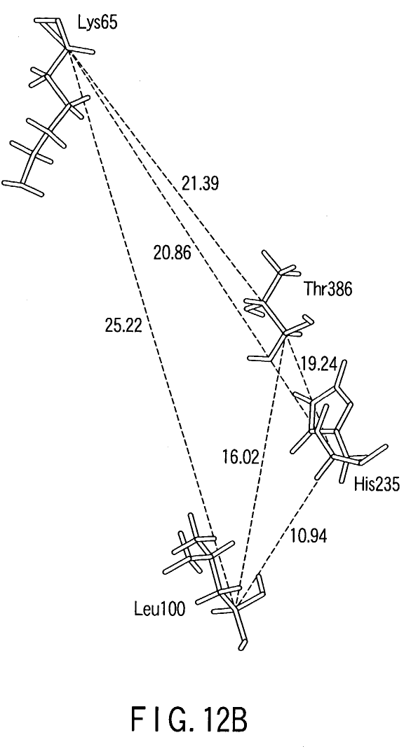

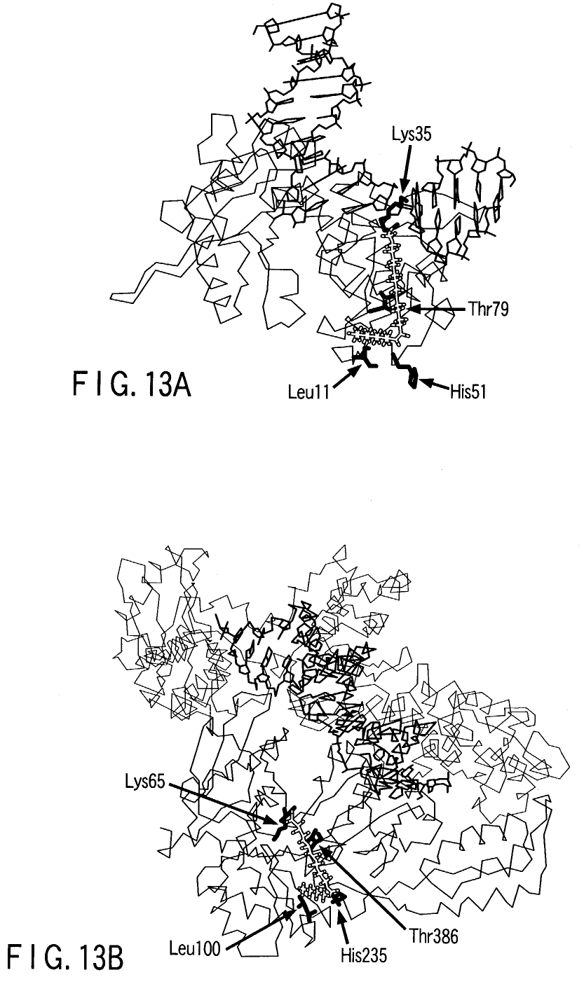

FIG. 12B shows, in the three-dimensional manner, the geometric configuration of the Cα atoms of Lys-65, Leu-100, His-235 and Thr-386. FIG. 13B shows a three-dimensional model of HIV reverse transcriptase to which nervonic acid has bound. For comparison, FIG. 12A and FIG. 13A show the geometric configuration and the three-dimensional model, respectively, of rat pol. β to which nervonic acid has bound.

<1-6> Study of the Binding Manner of Nervonic Acid on Rat Pol. β

FIGS. 14A and 14B show a three-dimensional molecular model of nervonic acid (NA) and rat pol. β, in which nervonic acid has bound on the 8 kDa domain of rat pol. β (prepared by with “Mol Graph”, manufactured by DAIKIN INDUSTRIES, Ltd.). FIG. 14B is a view in which the model of FIG. 14A has been turned by 90° around the vertical axis.

<1-7>

The confirmation of the inhibitory activity of nervonic acid on HIV reverse transcriptase was carried out.

Specifically, the inhibitory activity of nervonic acid on HIV reverse transcriptase was measured according to the method described in reference 8 listed below.

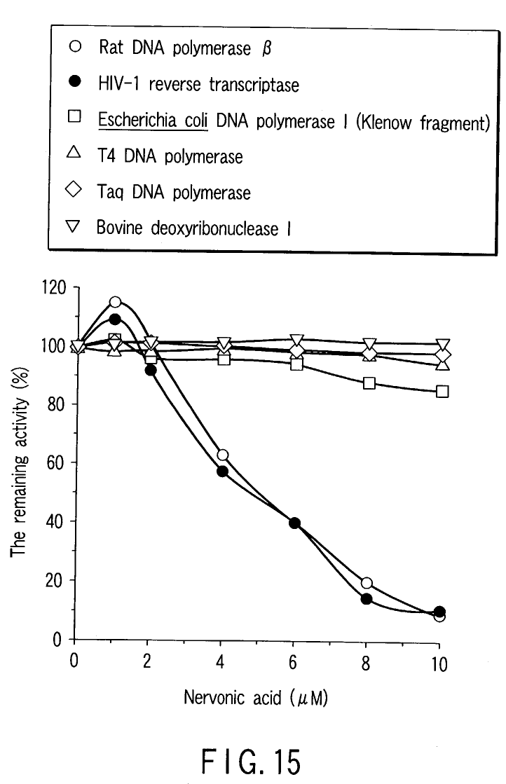

The result is shown in FIG. 15.

As is obvious from the inhibition curve shown in FIG. 15, the inhibitory activity of nervonic acid on HIV reverse transcriptase is substantially equal to the inhibitory activity of nervonic acid on rat pol. β.

<1-8> Designing an Inhibitor on HIV Reverse Transcriptase

As shown in the molecular models of FIGS. 13A and 13B of the <1-5>, it can be assumed that the binding manner of nervonic acid with rat pol. β is substantially the same as the binding manner of an inhibitor with HIV reverse transcriptase. Thus, it is possible to propose, as one example of the molecular structure of a preferable inhibitor on HIV reverse transcriptase, a cis-fatty acid in which the number of carbon atoms of the carbon chain is in the range of 16 to 24 and one double bond is located on the side of terminal methyl group with respect to the center of the carbon chain.

Further, designing a molecular structure as described below is also possible.

A fatty acid like nervonic acid is easily metabolized in vivo and therefore studying the role of rat pol. β is quite difficult in vivo. Thus, by modifying the molecular structure of the inhibitor such that the inhibitor will be non-metabolizable in vivo, while maintaining the molecular structure which presumably effects inhibitory activity on HIV reverse transcriptase, it is possible to provide a compound which maintains the inhibitory activity thereof in a body of a living organism in vivo.

### EXAMPLE 2

In this example, rat pol. β, yeast topo II, nervonic acid and human topo II were used as the first enzyme, the second enzyme, the first inhibitor and the third (target) enzyme, respectively, for designing a molecular structure of an inhibitor on human topo II.

<2-1> The geometric configuration of the binding sites of rat pol. β to which nervonic acid is to bind, was determined in a manner similar to that described in <1-4> of example 1.

<2-2> The Inhibitory Activity of Nervonic Acid on Yeast (S. cerevisiae) Topoisomerase II

The inhibitory activity of nervonic acid on yeast topo II was measured according to the method described in references 10 and 11.

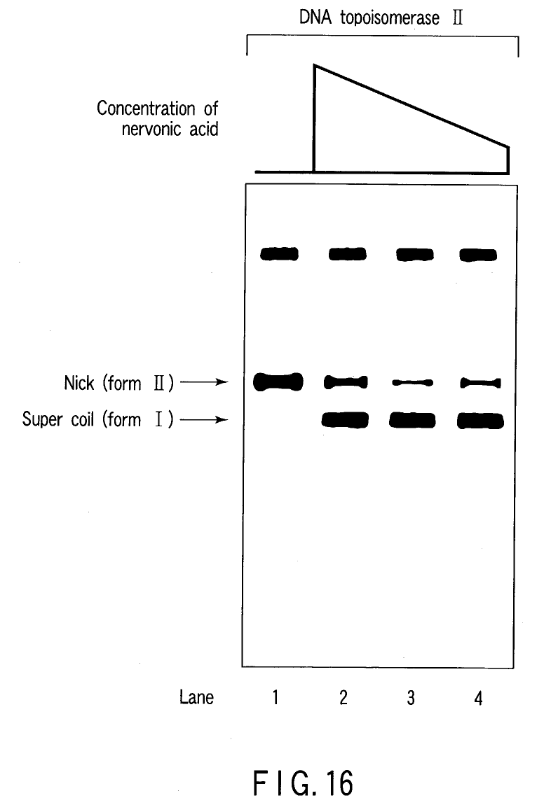

FIG. 16 shows the results of the measurement. FIG. 16 is a view showing the result of the electrophoresis in which lane 1 represents the absence of nervonic acid and other lanes each represent different concentrations of nervonic acid, which are 100 μM, 50 μM and 25 μM.

In FIG. 16, the nick (Form II) represents the band of the reaction product and the super coil (Form I) represents the band of the unreacted substance.

<2-3> Determination, by Searching, of the Target Amino Acid Group on Yeast Topo II

Whether such amino acids as those satisfying the following items 1) to 3) exist or not was searched using a computer software (INSIGHT II/BINDING SITE ANALYSIS, manufactured by MSI).

Item 1) determination, by searching, of whether or not a pocket amino acid group (yeast topo II pocket amino acid group-1) corresponding to the rat pol. β pocket amino acid group-1 is present in the amino acid sequence of yeast topo II;

Item 2) determination, by searching, of whether or not the amino acids constituting the yeast topo II pocket amino acid group-1 are present at the surface of yeast topo II in the tertiary structure thereof; and

Item 3) determination, by searching, of whether or not the amino acids constituting the yeast topo II pocket amino acid group-1 are conserved among topo II enzymes from different species, in terms of biological classification, from yeast.

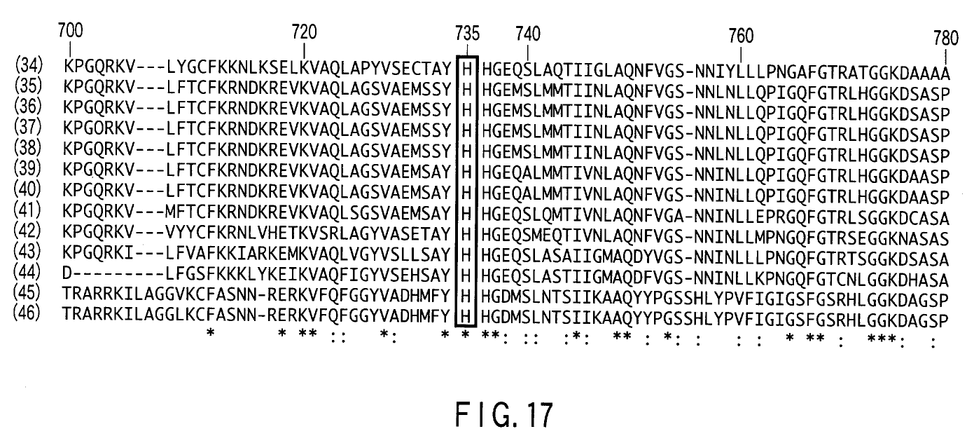

In the searching of the above item 1), the amino acids of yeast topo II having such distances between each of the Cα atoms as those within a range of ±2×10⁻¹ nm with respect to the corresponding distances between each of the Cα atoms in pol. β shown in Table 1, were selected. Further, in the search of the above item 3), 13 topo II enzymes derived from different species shown in FIG. 17 were used.

FIG. 17 shows a portion (amino acid Nos. 700 to 789) of the amino acid residues of 13 topo II enzymes, which correspond to SQE ID Nos. 34 to 46, respectively, of the Sequence Listing shown below.

From FIG. 17, it is revealed that His (H) of the amino acid No. 735 and Leu (L) of the amino acid No. 741 have been conserved, in terms of the evolution theory, in 13 topo II enzymes derived from different species.

As a result of the search, it was confirmed that Thr-596, His-735, Leu-741 and Lys-983 (the topo II pocket amino acid group-1) satisfy the above items 1) to 3).

FIG. 9B shows, in a three-dimensional manner, the geometric configuration of the Cα atoms of Thr-596, His-735, Leu-741 and Lys-983. Further, FIG. 10B indicates these four amino acid residues constituting the topo II pocket amino acid group-1, in a model in which the amino acid residues of topo II are expressed as ribbons.

FIG. 2B shows the amino acid sequence of yeast topo II (SEQ ID No. 2 of the Sequence Listing shown below). The amino acid sequence is available from SWISS-Plot Data Base: 1BJT.

When the sequence of yeast topo II shown in FIG. 2B is compared with the sequence of rat pol. β shown in FIG. 2A, the order in which Leu-11, Lys-35, His-51 and Thr-79 constituting the rat pol. β pocket amino acid group-1 appear in the primary sequence of the amino acid residues is different from the order in which Thr-596, His-735, Leu-741 and Lys-983 constituting the yeast topo II pocket amino acid group-1, except that the rat pol. β pocket amino acid group-1 and the topo II pocket amino acid group-1 have the same order of Leu and Lys therein. Further, the number of the amino acid residues present between each of the four amino acid residues is different, in the rat pol. β pocket amino acid group-1 and the topo II pocket amino acid group-1. Thus, it is very difficult to deduce the binding sites where nervonic acid is to bind to the two enzymes, from the primary sequences of the amino acid residues thereof.

### EXAMPLE 3

An experiment was conducted in a manner similar to that of example 2, except that lithocholic acid was used instead of nervonic acid as the first inhibitor.

<3-1> Measurements of ¹H-¹⁵N HMQC Spectroscopy of Rat Pol. β in the Presence and the Absence of Lithocholic Acid

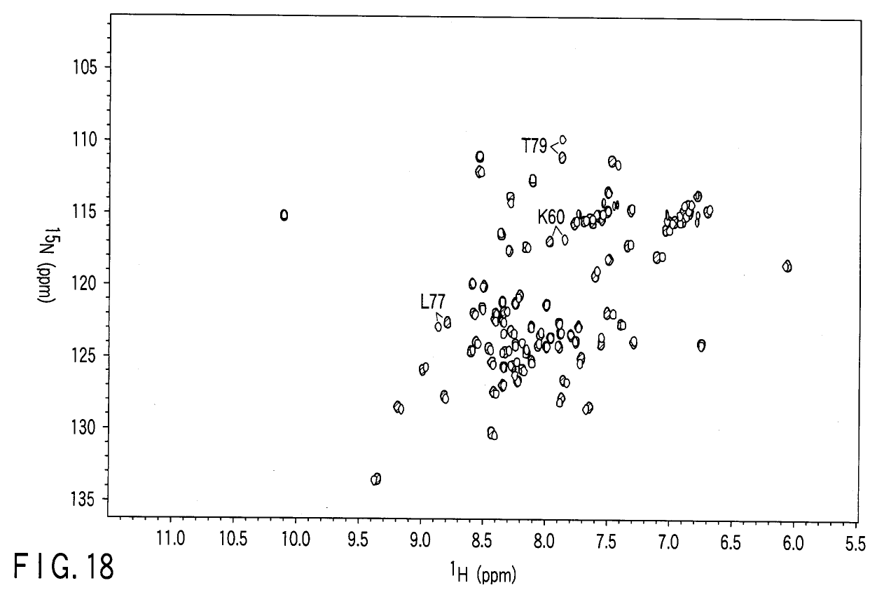

FIG. 18 shows the result of the ¹H-¹⁵N HMQC spectroscopy.

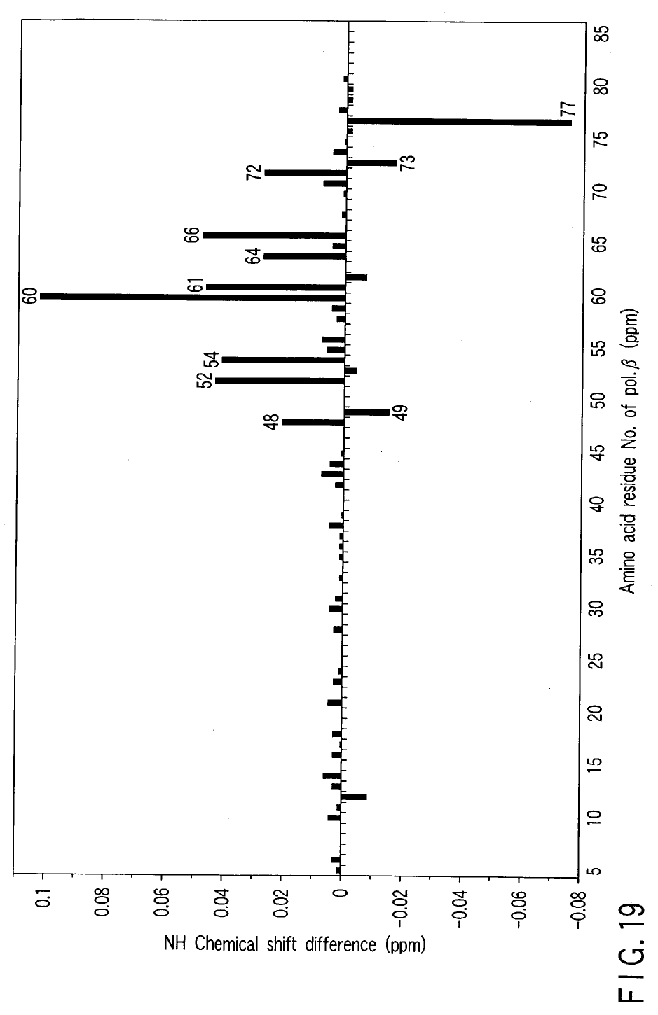

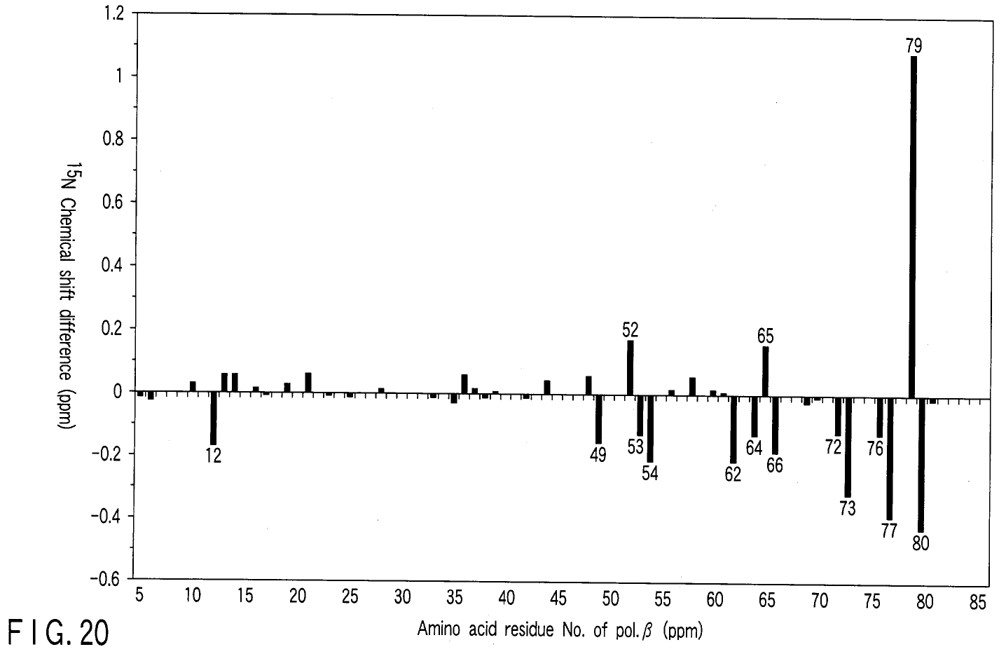

FIG. 19 is a bar graph in which the cross peaks of ¹H-¹⁵N HMQC spectroscopy shown in FIG. 18 is expressed as the relationship of ¹H chemical shift value vs. the amino acid residue number. FIG. 20 is a bar graph in which the cross peaks of ¹H-¹⁵N HMQC spectroscopy shown in FIG. 18 is expressed as the relationship of ¹⁵N chemical shift value vs. the amino acid residue number. In FIGS. 19 and 20, the vertical axis represents the chemical shift value and the horizontal axis represents the sequence number of the amino acid.

In this example, among the searched amino acid residues, those in which the absolute values of ¹H chemical shift and ¹⁵N chemical shift were 0.06 ppm or more and 0.6 ppm or more, respectively, were three amino acid residues: Lys-60, Leu-77 and Thr-79. The group consisting of these three amino acid residues will be referred to as “rat pol. β pocket amino acid group-2” hereinafter.

<3-2> Measurements of the Distances Between the Cα Atoms of Each of the Rat Pol. β Pocket Amino Acid Group-2

Table 3 shows the distances between the Cα atoms of the rat pol. β pocket amino acid group-2.

TABLE 3

| Distance between | |
|---|---|
| the Cα atoms of the | |
| amino acid residues | Distance (×10⁻¹ nm) |
| Lys-60 and Leu-77 | 14.97 |
| Lys-60 and Thr-79 | 19.67 |
| Leu-77 and Thr-79 | 5.70 |

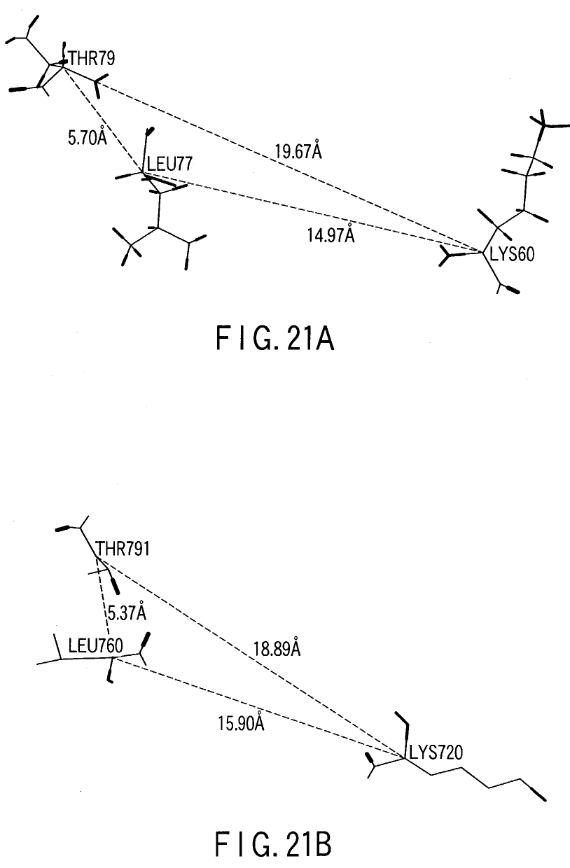

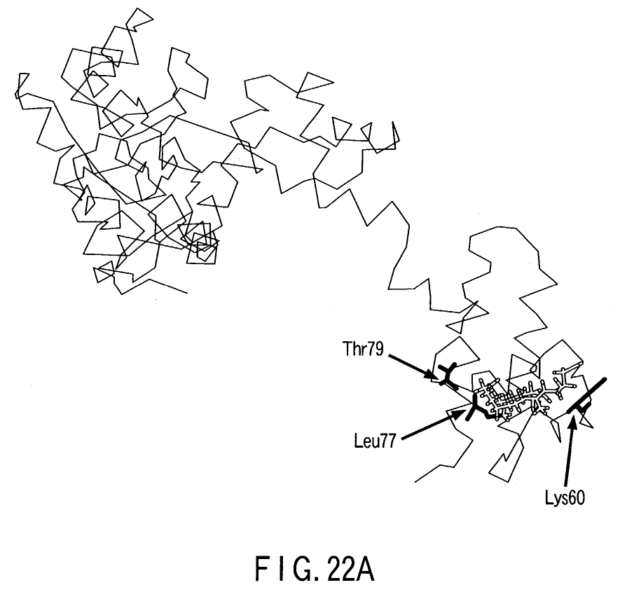

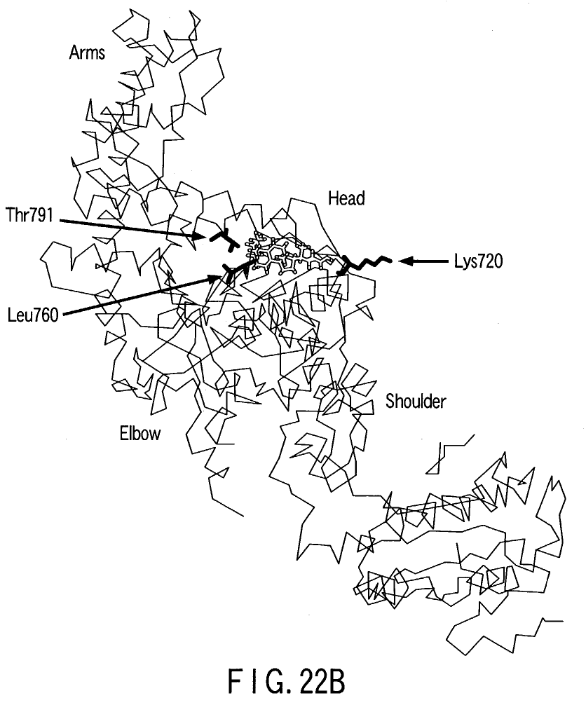

FIG. 21A is a view showing, in the three dimensional manner, the geometric configuration of the Cα atoms of the amino acid residues distanced from each other as shown in Table 3. FIG. 22A is a view showing, in the three dimensional manner, the amino acid residues of the rat pol. β, in a binding state with lithocholic acid, and the distances between the Cα atoms of these amino acid residues. Further, FIG. 22A is a view indicating the three amino acid residues constituting the rat pol. β pocket amino acid group-2, to which lithocholic acid has bound, in a model in which the amino acid residues of rat pol. β are expressed as a line.

<3-3> Determination, by Searching, of the Target Amino Acid Group in Yeast Topo II

Item 1) determination, by searching, of whether or not a pocket amino acid group (yeast topo II pocket amino acid group-2) corresponding to the rat pol. ! pocket amino acid group-2 are present in the amino acid sequence of yeast topo II;

Item 2) determination of whether or not the amino acids constituting the yeast topo II pocket amino acid group-2 are present at the surface of yeast topo II in the tertiary structure thereof; and

Item 3) determination, by searching, of whether or not the amino acids constituting the yeast topo II pocket amino acid group-2 are the amino acids conserved in other topo II enzymes derived from different species, in terms of biological classification, from yeast.

In the searching of the above item 1), the amino acids of yeast topo II having such distances between each of the Cα atoms as those within a range of +2×10⁻¹ nm with respect to the corresponding distance between each of the Cα atoms in pol. β shown in Table 3, were selected.

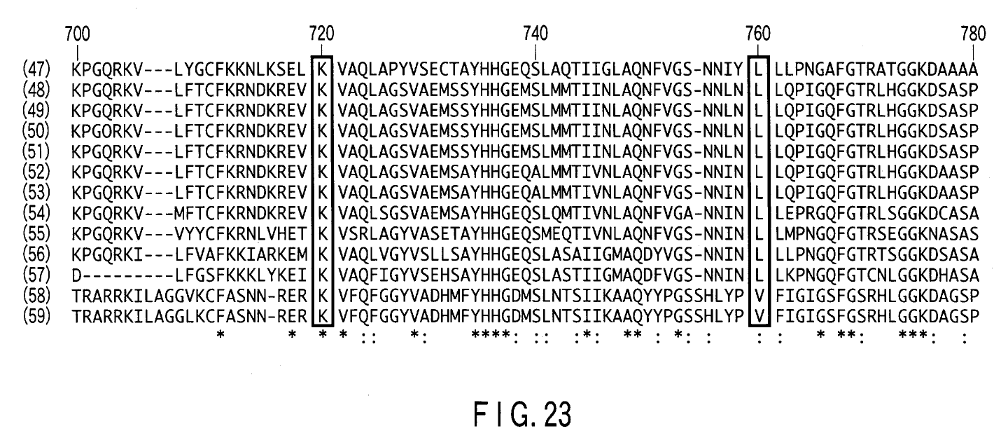

FIG. 23 shows a portion (amino acid Nos. 700 to 780) of the amino acid residues of topo II of 13 types which correspond to SQE ID Nos. 34 to 46, respectively, of the Sequence Listing shown below.

From FIG. 23, it is understood that Lys (K) of the amino acid No. 720 and Leu (L) of the amino acid No. 760 have been conserved, in terms of the evolution theory, in 13 topo II enzymes derived from different species.

As a result of the search, it was confirmed that Lys-720, Leu-760 and Thr-791 (topo II pocket amino acid group-2) satisfy the above items 1) to 3).

FIG. 21B shows, in a three-dimensional manner, the geometric configuration of the Cα atoms of Lys-720, Leu-760 and Thr-791. Further, FIG. 22B indicates these three amino acid residues constituting the topo II pocket amino acid group-2, to which lithocholic acid has bound, in a model in which the amino acid residues of topo II are expressed as a line.

<3-4> Designing a Novel Inhibitor of Human Topo II

In consideration of the binding manner of lithocholic acid to rat pol. β described in the <2-6>, it is expected that a steroid skeleton having at least a carboxyl group in the vicinity of the terminal end of the molecular structure thereof will advantageously be used as the structure of an inhibitor of human topo II. Further, it will also be advantageous to add a side chain such as an alkyl group to the molecular structure, such that the inhibitor is to bind more fittingly to the triangle formed by Lys-720, Leu-760 and Thr-791.

### REFERENCES

The entire contents disclosed in the following references are incorporated herein by reference.

(1) Mizushina et al., J. Antibiot. (Tokyo) 49, 491-492 (1996)

(2) Mizushina et al., Biochem. Biophys. Acta 1308, 256-262 (1996)

(3) Mizushina et al., Biochem. Biophys. Acta 1336, 509-521 (1997)

(4) Mizushina et al., Biol. Chem. 274, 25599-25607 (1999)

(5) B. Z. Zmudzka et al., Proc. Natl. Acad. Sci. U.S.A., 83, 5106-5110 (1986)

(6) H. Pelletier et al., Science, 264, 1891-1903 (1994)

(7) M. R. Sawaya et al., Science, 264, 1930-1935 (1994)

(8) Mizushina et al., Biochem. Biophys. Acta, 1336, 509-521 (1997)

(9) D. Liu et al., Biochemistry, 33, 9537-9545 (1994)

(10) F. Liu et al., Proc. Natl. Acad. Scie. USA 78, 3487-3491 (1981)

(11) A. Ferrp et al., J. Biol. Chem. 259, 547-554 (1984)

(12) D. Liu et al., Biochemistry, 35, 6188-6200 (1996)

(13) R. Prasad et al., J. Biol. Chem., 273, 111121-11126 (1998)

(14) Kobayashi et al., Nature Struct. Biol. 4, 677 (1997)

(15) Wallace et al., Protein Sci. 6, 2308-2323 (1997)

(16) Russell et al., J. Mol. Biol. 279, 1211-1227 (1998)

What is claimed is:

1. A method of designing a molecular structure of an inhibitor on a target enzyme whose secondary and tertiary structures are known, by
analyzing, in the tertiary structure of a first enzyme whose primary, secondary and tertiary structures are known, a geometric configuration formed by amino acids to which a first inhibitor, having biological, inhibitory activity on the first enzyme, is to bind; and
searching, in the target enzyme, the similar geometric configuration to the geometric configuration that was analyzed in the first enzyme;
thereby designing the molecular structure of the inhibitor on the target enzyme, wherein the method comprises:
(1) determining, in the tertiary structure of the first enzyme, a first pocket amino acid group consisting of plural amino acids to which the first inhibitor is to bind, wherein the plural amino acids are determined by comparing ¹H-¹⁵N HMQC spectroscopy of the first enzyme in the absence of the first inhibitor with ¹H-¹⁵N HMQC spectroscopy of the first enzyme in the presence of the first inhibitor, and selecting amino acids that satisfy the condition that the absolute value of ¹H chemical shift is equal to or more than a specific value and/or the absolute value of ¹⁵N chemical shift is equal to or more than another specific value;
(2) determining the geometric configuration of the first pocket amino acid group, by measuring, in the tertiary structure of the first enzyme, distances between the Cα atoms of each of the plural amino acids constituting the first pocket amino acid group;
(3) detecting, in the tertiary structure of the target enzyme, one or more candidates for a target pocket amino acid group having a geometric configuration similar to the geometric configuration formed by the first pocket amino acid group determined in (2), wherein it is presumed that the candidate for the target pocket amino acid group exists on the target enzyme if the following conditions are satisfied:
respective amino acids constituting the candidate for the target pocket amino acid group are of the same kinds as the plural amino acids constituting the first pocket amino acid group determined in (2), and
the absolute values of differences between (a) and (b) are within a specific value, wherein (a) is respective distances, in the tertiary structure of the target enzyme, between the Cα atoms of each of the amino acids constituting the target pocket amino acid group, and (b) is respective distances between the Cα atoms in the first pocket amino acid group obtained in (2); and
(4) screening the amino acids constituting the candidate for the target pocket amino acid group by the following two requirements:
(requirement 1) the amino acids are those present at the surface of the target enzyme in the tertiary structure thereof; and
(requirement 2) the amino acids are those that have been conserved among species, when a comparison is made between the primary sequence of the amino acids of the target enzyme and the primary sequence of the amino acids of another enzyme having the same biochemical activity as the target enzyme but derived from an organism of a different species from that of the target enzyme, in terms of biological classification; and
determining the amino acids satisfying the above two requirements to be those constituting the usable target pocket amino acid group for designing the molecular structure of the inhibitor on the target enzyme; thereby
the molecular structure of the inhibitor on the target enzyme is designed on the basis of the kinds of amino acid residues of the first enzyme, to which the first inhibitor is to bind, and the geometric configuration of the amino acid residues.

2. The method according to claim 1, wherein the first enzyme is rat DNA polymerase β, the first inhibitor is nervonic acid and the target enzyme is human immunodeficiency virus reverse transcriptase;
in said determining amino acids of (1), the amino acids satisfying the absolute value of ¹H chemical shift of 0.06 ppm or more, or the absolute value of ¹⁵N chemical shift of 0.4 ppm or more, are selected thereby a rat DNA polymerase β pocket amino acid group is determined to be Leu-11, Lys-35, His-51 and Thr-79;
in said determining the geometric configuration of (2), the geometric configuration of a triangular pyramid formed by Leu-11, Lys-35, His-51 and Thr-79 is determined;
in said detecting the tertiary structure of (3), the values of the differences between the respective distances of Cα atoms of each of Leu, Lys, His and Thr in the tertiary structure of human immunodeficiency virus reverse transcriptase and the respective distances of Cα atoms obtained in (2), are set to be ±2×10⁻¹ nm, thereby determining the candidate for the target pocket amino acid group;
in said screening of (4), the usable target pocket amino acid group for designing the molecular structure of the inhibitor is determined to be Lys-65, Leu-100, His-235 and Thr-386 of human immunodeficiency virus reverse transcriptase; and
thereby the molecular structure of the inhibitor on human immunodeficiency virus reverse transcriptase is designed on the basis of the kinds of amino acid residues of rat DNA polymerase β to which nervonic acid is to bind, and the geometric configuration of the amino acid residues.

3. A method of designing a molecular structure of an inhibitor on a target enzyme whose tertiary structure is unknown, by
analyzing, in the tertiary structure of a first enzyme whose primary, secondary and tertiary structures are known, a geometric configuration formed by amino acids to which a first inhibitor, having biological, inhibitory activity on the first enzyme, is to bind; and
searching, in a second enzyme whose primary, secondary and tertiary structures are known, the similar geometric configuration to the geometric configuration that was analyzed in the first enzyme;
thereby designing the molecular structure of the inhibitor on the target enzyme,
wherein the method comprises:
(1) determining, in the tertiary structure of the first enzyme, a first pocket amino acid group consisting of plural amino acids to which the first inhibitor is to bind, wherein the plural amino acids are determined by comparing ¹H-¹⁵N HMQC spectroscopy of the first enzyme in the absence of the first inhibitor with ¹H-¹⁵N HMQC spectroscopy of the first enzyme in the presence of the first inhibitor, and selecting amino acids that satisfy the condition that the absolute value of ¹H chemical shift is equal to or more than a specific value and/or the absolute value of ¹⁵N chemical shift is equal to or more than another specific value;
(2) determining the geometric configuration of the first pocket amino acid group, by measuring, in the tertiary structure of the first enzyme, the distances between the Cα atoms of each of the plural amino acids constituting the first pocket amino acid group;
(3) detecting, in the tertiary structure of the second enzyme, a second pocket amino acid group having a geometric configuration similar to the geometric configuration formed by the first pocket amino acid group determined in (2), wherein it is presumed that the second pocket amino acid group has a geometric configuration similar to the geometric configuration formed by the first pocket amino acid group if the following requirements are satisfied:
(requirement 1) the second enzyme has the same biochemical activity as that of the target enzyme;
(requirement 2) the organism from which the second enzyme is derived, belongs to a different species, in terms of biological classification, from the organism from which the target enzyme is derived;
(requirement 3) the primary, secondary and tertiary structures of the second enzyme are known; and
(requirement 4) the second enzyme is biochemically inhibited by the first inhibitor, and
respective amino acids constituting the second pocket amino acid group are of the same kinds as the plural amino acids constituting the first pocket amino acid group determined in (2), and
the absolute values of differences between (a) and (b) are within a specific value, wherein (a) is respective distances, in the tertiary structure of the second enzyme, between the Cα atoms of each of the amino acids constituting the second pocket amino acid group, and (b) is respective distances between the Cα atoms in the first pocket amino acid group obtained in (2); and
(4) screening the amino acids constituting the second pocket amino acid group by the following two requirements:
(requirement 1) the amino acids are those present at the surface of the second enzyme in the tertiary structure thereof; and
(requirement 2) the amino acids are those that have been conserved among species, when a comparison is made among the primary sequence of the amino acids of the second enzyme, the primary sequence of the amino acid of the target enzyme and the primary sequence of the amino acids of another enzyme having the same biochemical activity as that of the second enzyme but derived from an organism of a different species from that of both the second enzyme and the target enzyme, in terms of biological classification; and
determining the amino acids satisfying the above two requirements to be those constituting the usable target pocket amino acid group for designing the molecular structure of the inhibitor on the target enzyme; thereby
the molecular structure of the inhibitor on the target enzyme is designed on the basis of the kinds of amino acid residues of the first enzyme, to which the first inhibitor is to bind, and the geometric configuration of the amino acid residues.

4. The method according to claim 3, wherein the first enzyme is rat DNA polymerase β, the first inhibitor is nervonic acid, the second enzyme is yeast DNA topoisomerase II and the target enzyme is human DNA topoisomerase II;
in said determining amino acids of (1), the amino acids satisfying the absolute value of ¹H chemical shift of 0.06 ppm or more, or the absolute value of ¹⁵N chemical shift of 0.4 ppm or more, are selected thereby a rat DNA polymerase P pocket amino acid group is determined to be Leu-11, Lys-35, His-51 and Thr-79;
in said determining the geometric configuration of (2), the geometric configuration of a triangular pyramid formed by Leu-11, Lys-35, His-51 and Thr-79 is determined;
in said detecting the tertiary structure of (3), the values of the differences between the respective distances of Cα atoms of each of Leu, Lys, His and Thr in the tertiary structure of yeast DNA topoisomerase II and the respective distances of Cα atoms obtained in (2), are set to be ±2×10⁻¹ nm, thereby determining the yeast DNA topoisomerase II pocket amino acid group;
in said screening of (4), the usable target pocket amino acid group for designing the molecular structure of the inhibitor is determined to be Thr-596, His-735, Leu-741 and Lys-983 of yeast DNA topoisomerase II; and
thereby the molecular structure of the inhibitor on human DNA topoisomerase II is designed on the basis of the kinds of amino acid residues of yeast DNA topoisomerase II, to which nervonic acid is to bind, and the geometric configuration of the amino acid residues.

5. The method according to claim 3, wherein the first enzyme is rat DNA polymerase β, the first inhibitor is lithocholic acid, the second enzyme is yeast DNA topoisomerase II and the target enzyme is human DNA topoisomerase II;
in said determining amino acids of (1), the amino acids satisfying the absolute value of ¹H chemical shift of 0.06 ppm or more, or the absolute value of ¹⁵N chemical shift of 0.4 ppm or more, are selected thereby a rat DNA polymerase β pocket amino acid group is determined to be Lys-60, Leu-77 and Thr-79;
in said determining the geometric configuration of (2), the geometric configuration of a triangle formed by Lys-60, Leu-77 and Thr-79 is determined;
in said detecting the tertiary structure of (3), the values of the differences between the respective distances of Cα atoms of each of Lys, Leu and Thr in the tertiary structure of yeast DNA topoisomerase II and the respective distances of Cα atoms obtained in (2), are set to be ±2×10⁻¹ nm, thereby determining the yeast DNA topoisomerase II pocket amino acid group;
in said screening of (4), the usable target pocket amino acid group for designing the molecular structure of the inhibitor is determined to be Lys-720, Leu-760 and Thr-791 of yeast DNA topoisomerase II; and
thereby the molecular structure of the inhibitor on human DNA topoisomerase II is designed on the basis of the kinds of amino acid residues of yeast DNA topoisomerase II, to which lithocholic acid to bind, and the geometric configuration of the amino acid residues.
